(19) United States
(12) Patent Application Publication
(10) Pub. No.: US 2004/0039334 A1
(43) Pub. Date: Feb. 26, 2004

(54) Self-destructing syringe

(76) Inventor: Sai-Ling Li, Hunan (CN)

Correspondence Address: BROWDY AND NEIMARK, P.L.L.C., 624 NINTH STREET, NW, SUITE 300, WASHINGTON, DC, 20001-5303, US

(21) Appl. No.: 10/333,825

(22) PCT Filed: Jul. 25, 2001
(86) PCT No.: PCT/CN01/01205

(30) Foreign Application Priority Data
Jul. 25, 2000 (CN) 00243818.6
Nov. 6, 2000 (CN) 00259762.4
Dec. 25, 2000 (CN) 00267535.8
Dec. 25, 2000 (CN) 00267534.X

(51) Int. Cl. A61M 5/00

(52) U.S. Cl. 604/110; 604/192

(57) ABSTRACT

A self-destructing syringe includes an elliptic needle cylinder, a plunger, a needle holder and a needle. A handle is provided on one end of the plunger, and a piston is provided on the other end of the plunger. The handle is engaged with the piston by a plunger body. There is a hole on the bottom center of the needle cylinder, which is used to mount the needle holder. The holder is mounted on the needle cylinder by the piston located on the bottom of the needle cylinder. The plunger body has a breakable region. The needle cylinder and the needle cap are incorporate, on which a disposable destructing device is provided.

### FIELD OF THE INVENTION

The present invention relates to a disposable medical instrument and, more particularly, to a self-destructing syringe.

### BACKGROUND OF THE INVENTION

Most existent syringes comprise a needle cylinder, a plunger and an injection needle. The needle cylinder is of a cylindrical shape. The needle can be separately slipped onto a needle connector, which sticks out from bottom of the needle cylinder. One end of the plunger has a piston, which is used to inject injection liquid in the needle cylinder into a human body through the needle connector and the needle. The other end of the plunger has a handle. The end portion of the needle cylinder has also an expanded hand support. Through the handle and the hand support, medicinal liquid in the needle cylinder will be injected into the human body. However, this kind of syringe has the following drawbacks in use.

1. Because medical treatment is a serious job for prevention and cure of disease, prevention of disease spreading, especially infection due to direct contact with viruses, allows of no disregard. There are usually some lawless persons who recycle or collect used disposable syringes and then sell out after repackaging for their own benefits. Because existent syringes have no mandatory destructing devices, lawless persons have loopholes to exploit.

2. For existent syringes, because the needle is mounted on the needle cylinder thereof after use, the exposed needle head may easily stab the human body to cause defection, especially the medical personnel and persons for post processing.

### SUMMARY OF THE INVENTION

An object of the present invention is to provide a novel syringe, which can destruct its components one by one during use to effectively avoid illegal recycling, can retract the needle head to avoid spread of disease sources, and has a convenient and reliable usage.

To achieve the above object, the present invention provides a syringe, which includes an elliptic needle cylinder, a plunger, a plunger hanger, a hanger cavity, a needle holder and a needle. A handle is provided on one end of the plunger. Symmetric protection wings are provided on one end of the handle. A piston is provided on the other end of the plunger. The handle is engaged with the piston by a plunger body. The front end of the plunger passes through a central hole of the piston, and is engaged with the plunger hanger through a thread. There is a hole at the bottom center of the needle cylinder, which is used to mount the needle holder. The needle holder and the hanger cavity screwed therewith are mounted on the needle cylinder by the piston located on the bottom of the needle cylinder. The needle cylinder and the needle cap are incorporate, between which a breakable and destructible region is provided. The needle and the needle holder are incorporate. The cross section of the plunger body is a crossed structure. The breakable region on the plunger body is openings on four faces of the crossed structure at the middle of the plunger body or breakable grooves at predetermined portions of the plunger body. A destructing device on the plunger body is accomplished by providing a weak ring on the elliptic piston holder.

To achieve the above object, the present invention also provides a syringe, which includes an elliptic needle cylinder, a plunger, a plunger hanger, a hanger cavity, a needle holder and a needle holder lock. A handle is provided at one end of the plunger. Symmetrical protection wings are provided at one end of the handle. A piston is provided at the other end of the plunger. The handle is engaged with the piston by the plunger body. There is a hole at the bottom center of the needle cylinder, which is used to mount the needle holder. The needle holder and the hanger cavity screwed therewith are mounted on the needle cylinder by the piston located on the bottom of the needle cylinder. The plunger body has a breakable and destructible region. The needle cylinder and the needle cap are incorporate. The cross section of the plunger body is a crossed structure. The breakable region on the plunger body is openings on the four faces of the crossed structure at the middle of the plunger body or breakable grooves at predetermined portions of the plunger body. A destructing device on the plunger body is accomplished by providing a weak ring on the elliptic piston holder. In order to let the destructing device be effective, a gap is provided at the connection position of the needle plunger and the piston holder. The gap is engaged with the inner circle of the ring-shaped destructing device.

To achieve the above object, the present invention also provides a syringe, which comprises an elliptic needle cylinder, a plunger, a plunger hanger, a hanger cavity, a needle holder and a needle holder lock. A handle is provided at one end of the plunger. Symmetrical protection wings are provided at one end of the handle. A piston is provided at the other end of the plunger. The handle is engaged with the piston by the plunger body. There is a hole at the bottom center of the needle cylinder, which is used to mount the needle holder lock. The needle holder lock and the hanger cavity whose rear is screwed therewith are mounted on the needle cylinder by the needle holder piston. The needle holder and the hanger cavity are connected together through a thread, and are mounted on the needle cylinder by the needle holder piston. The needle cap and the needle holder are incorporate. The plunger body has a breakable region. The cross section of the plunger body is a crossed structure. A destructing device on the plunger body is accomplished by providing a weak ring on the elliptic piston holder. A gap is provided at the connection position of the needle plunger and the piston holder. The gap is engaged with the inner circle of the ring-shaped destructing device.

To achieve the above object, the present invention also provides a syringe, which comprises an elliptic needle cylinder, a plunger, a needle holder and a needle. A handle is provided at one end of the plunger. Symmetrical protection wings are provided at two ends of the handle. A piston is provided at the other end of the plunger. The handle is engaged with the piston by the plunger body. There is a hole at the bottom center of the needle cylinder, which is used to mount the needle holder. The needle holder is engaged with the needle cylinder by the piston located on the bottom of the needle cylinder. The plunger body has a breakable region. The cross section of the plunger body is a crossed structure. The breakable region on the plunger body is openings on the four faces of the crossed structure at the middle of the plunger body or breakable grooves at predetermined portions of the plunger body. The needle is tightly engaged with the needle holder by a connection structure located at the front end of the needle holder. The needle cap and the needle cylinder are incorporate, between which a weak destructing device provided is provided.

The various objects and advantages of the present invention will be more readily understood from the following detailed description when read in conjunction with the appended drawings, in which:

### BRIEF DESCRIPTION OF THE DRAWINGS

FIG. 1 is a whole structure diagram of the present invention;

FIG. 2 shows structures of a needle cylinder and a needle cap of the present invention, wherein FIG. 2a is a main diagram, FIG. 2b is a perspective view along line B-B and FIG. 2c is a perspective view along line A-A;

FIG. 3 shows a structure of a plunger of the present invention, wherein FIG. 3a is a main diagram, FIG. 3b is a right side view, FIG. 3c is a left side view and FIG. 3d is a transversally cross-sectional view;

FIG. 4 shows a structure of a plunger hanger of the present invention, wherein FIG. 4a is a main diagram, FIG. 4b is a left side view, FIG. 4c is a top view, FIG. 4d is a right side view and FIG. 4e is a cross-sectional view along line A-A;

FIG. 5 shows a structure of a plunger piston of the present invention, wherein FIG. 5a is a main-diagram, FIG. 5b is a top view and FIG. 5c is a bottom view;

FIG. 6 shows a structure of a needle holder piston of the present invention, wherein FIG. 6a is a main diagram, FIG. 6b is a right side view and FIG. 6c is a left side view;

FIG. 7 shows a structure of a hanger cavity of the present invention, wherein FIG. 7a is a main diagram, FIG. 7b is a left side view, FIG. 7c is a right side view, FIG. 7d is a perspective view along line A-A and FIG. 7e is a top view;

FIG. 8 shows a structure of a needle holder of the present invention, wherein FIG. 8a is a main diagram, FIG. 8b is a right side view and FIG. 8c is a top view;

FIG. 9 shows another structure of a needle plunger of the present invention, wherein FIG. 9a is a main diagram, FIG. 9b is a right side view, FIG. 9c is a top view, FIG. 9d is a cross-sectional view along line A-A and FIG. 9e is a cross-sectional view along line B-B;

FIG. 10 shows another structure of a plunger hanger of the present invention, wherein FIG. 10a is a main diagram, FIG. 10b is a left side view, FIG. 10c is a right side view and FIG. 10d is a bottom view;

FIG. 11 shows another structure of a plunger piston of the present invention, wherein FIG. 11a is a main diagram, FIG. 11b is a left side view and FIG. 11c is a right side view;

FIG. 12 shows another structure of a needle holder piston of the present invention, wherein FIG. 12a is a main diagram, FIG. 12b is a left side view and FIG. 12c is a right side view;

FIG. 13 shows another structure of a hanger cavity of the present invention, wherein FIG. 13a is a main diagram, FIG. 13b is a left side view, FIG. 13c is a right side view, FIG. 13d is a top view and FIG. 13e is a cross-sectional view along line A-A;

FIG. 14 shows another structure of a needle holder of the present invention, wherein FIG. 14a is a main diagram, FIG. 14b is a right side view and FIG. 14c is a top view;

FIG. 15 shows structures of a needle cylinder and a needle cap of the present invention, wherein FIG. 15a is a main diagram, FIG. 15b is a perspective view along line B-B and FIG. 15c is a perspective View along line A-A;

FIG. 16 shows yet another structure of a plunger hanger of the present invention, wherein FIG. 16a is a main diagram, FIG. 16b is a left side view, FIG. 16c is a right side view and FIG. 16d is a bottom view;

FIG. 17 shows yet another structure of a hanger cavity of the present invention, wherein FIG. 17a is a main diagram, FIG. 17b is a left side view, FIG. 17c is a right side view, FIG. 17d is a cross-sectional view along line A-A and FIG. 17e is a top view;

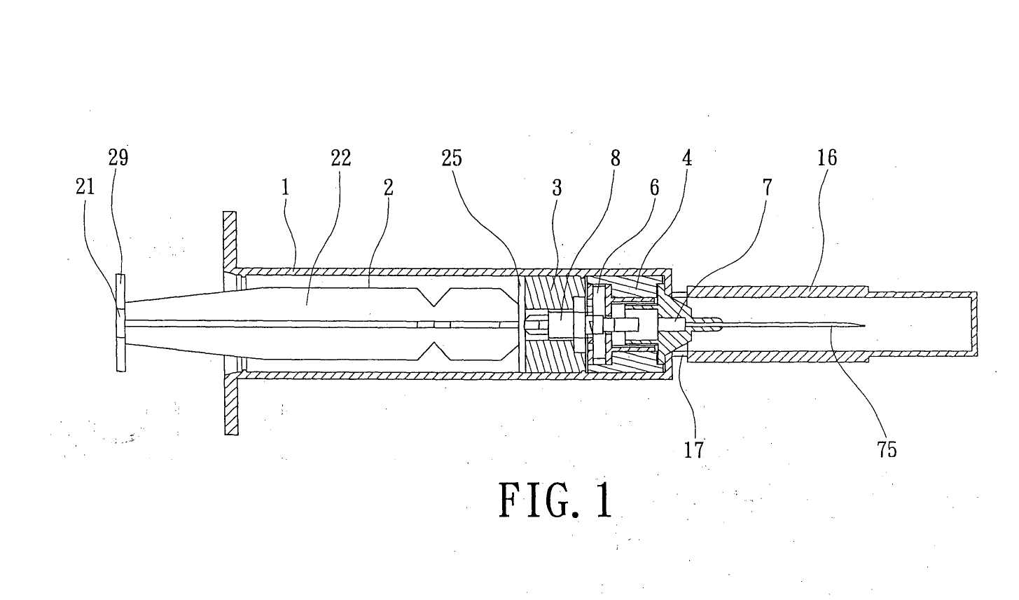

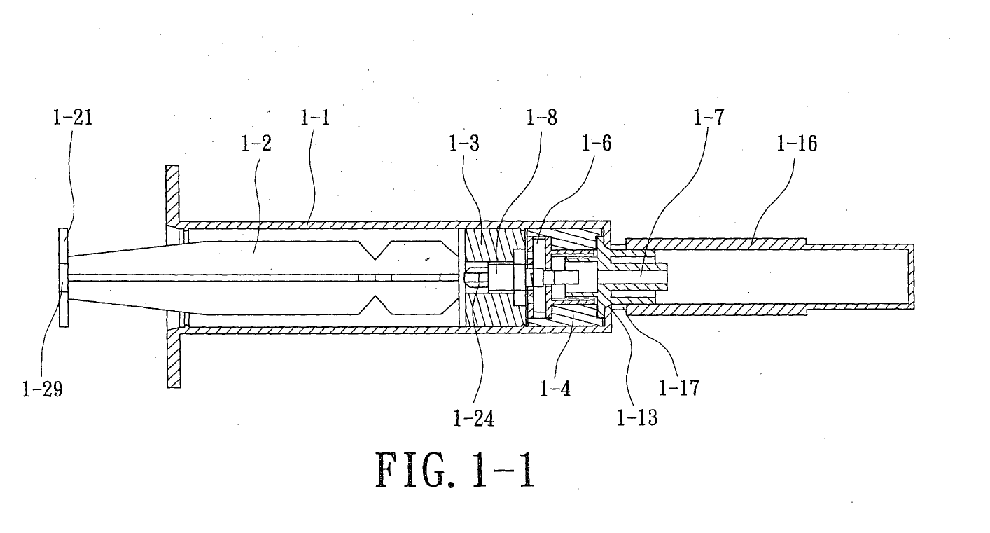

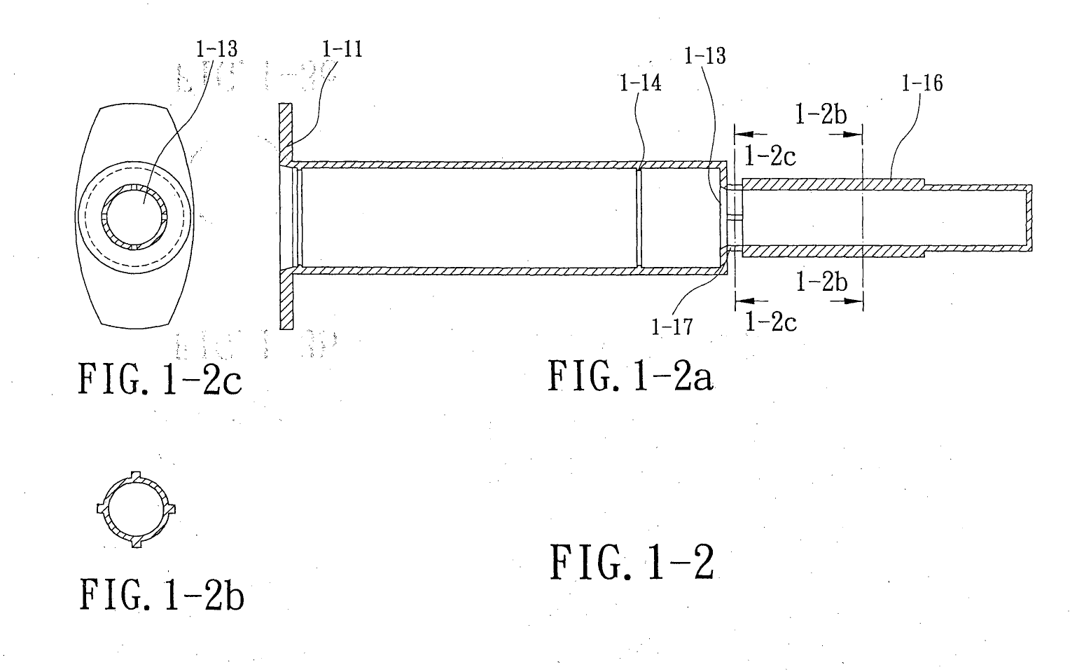

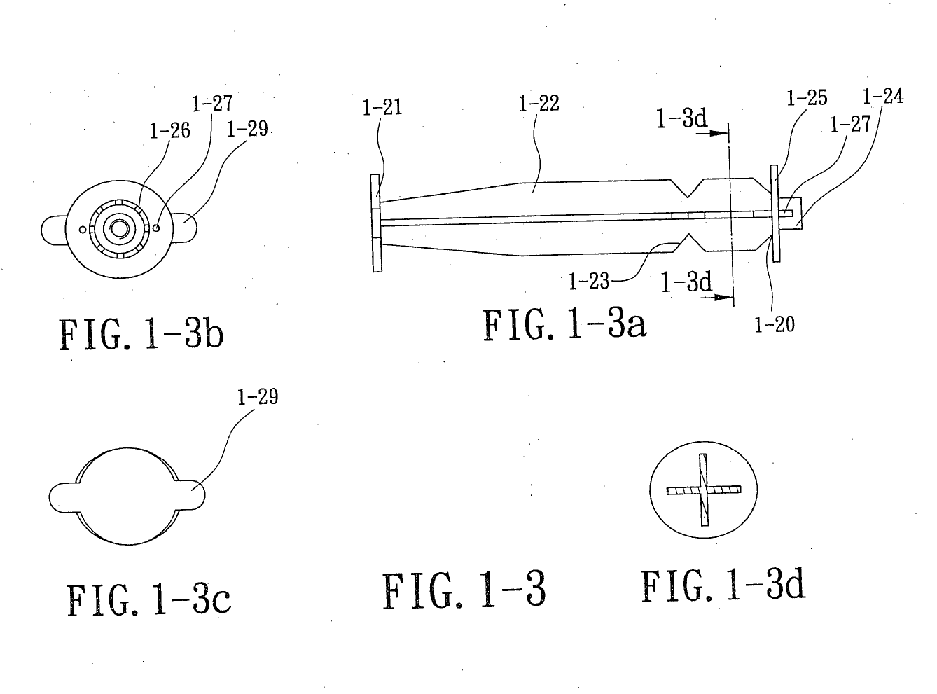

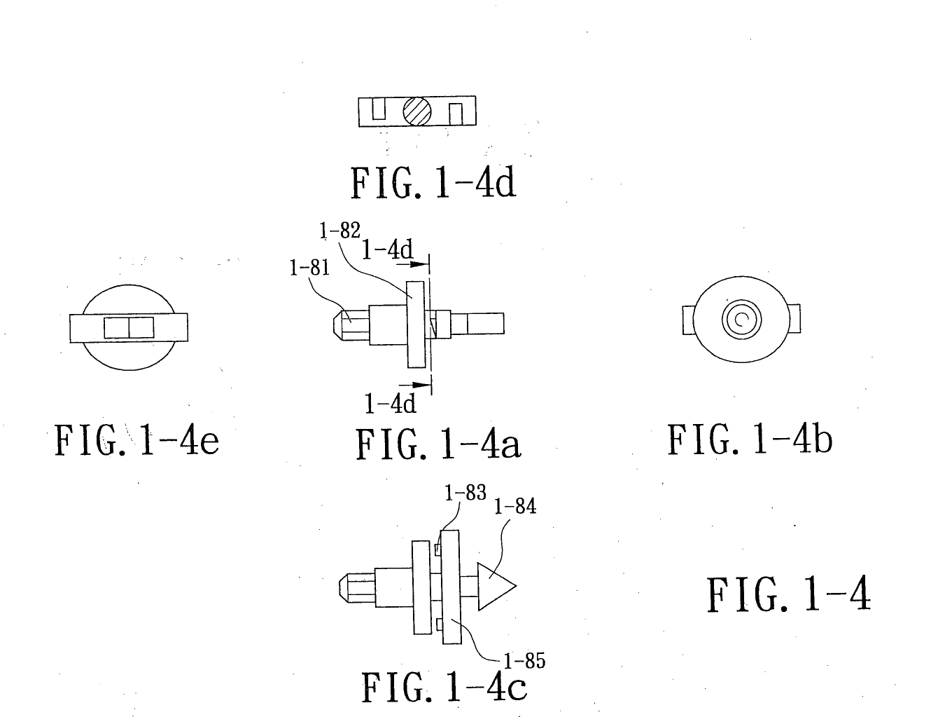

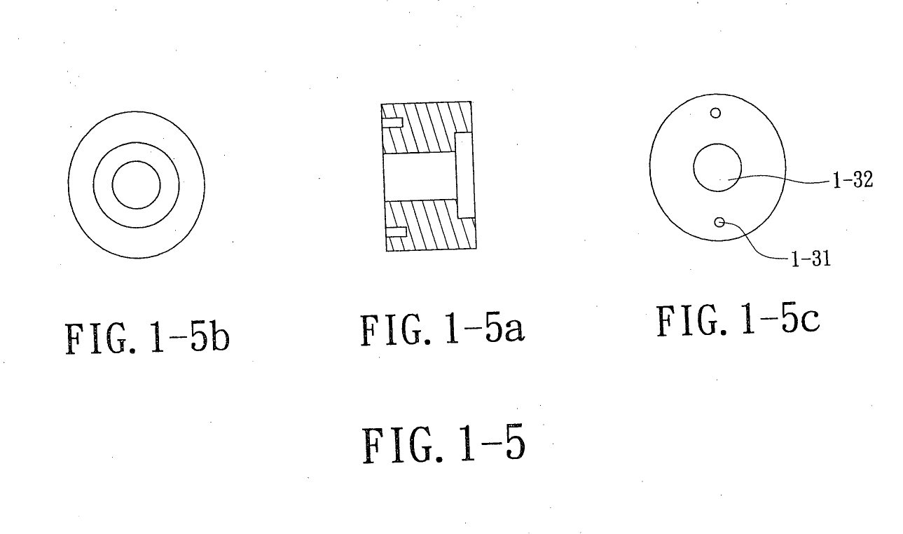

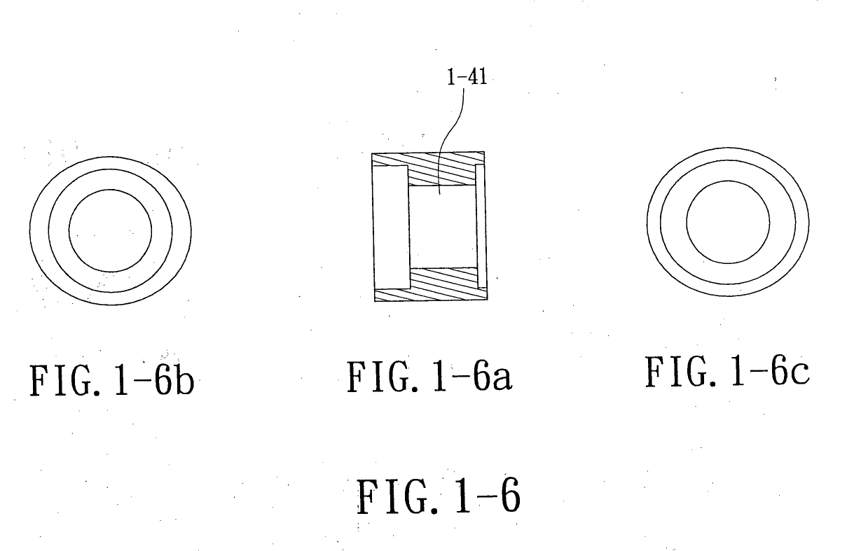

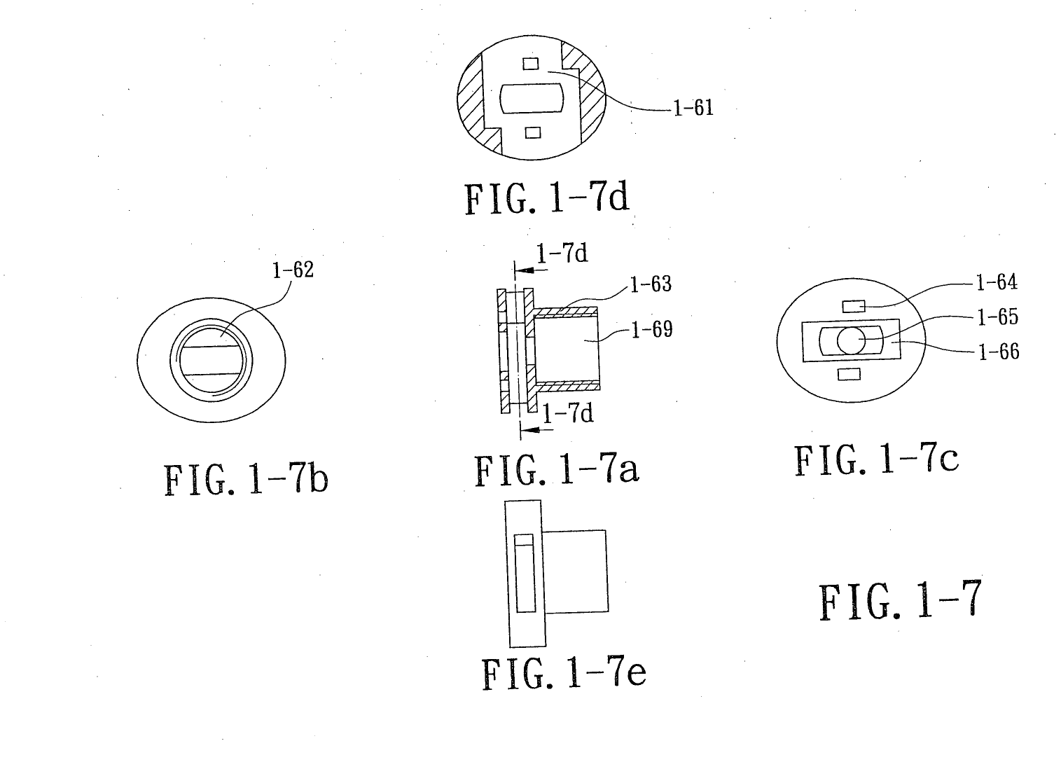

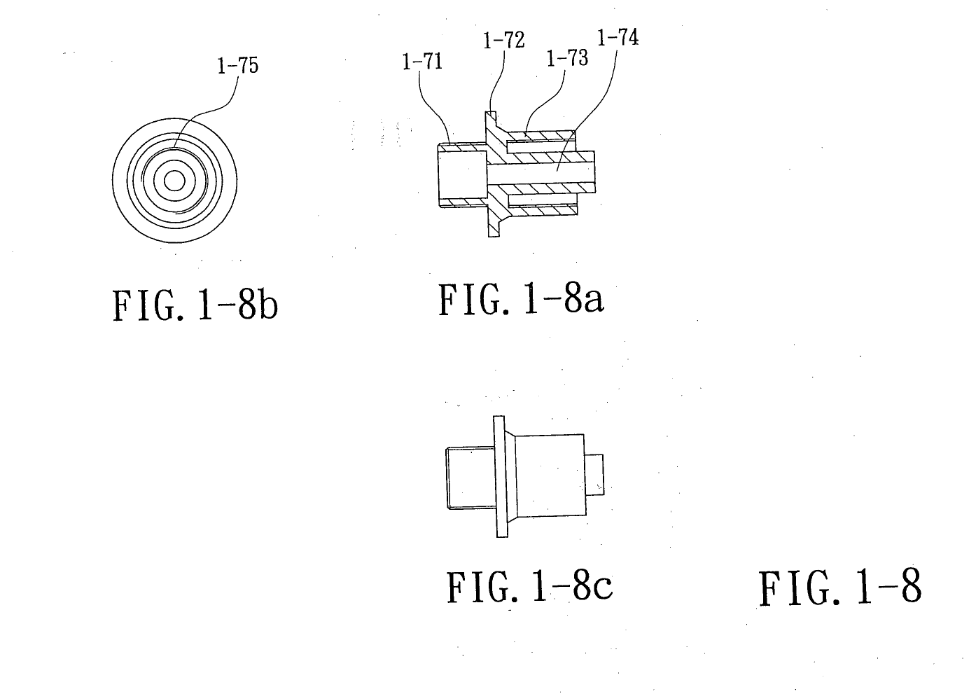

FIG. 1-1 is a whole structure diagram according to a first embodiment of the present invention;

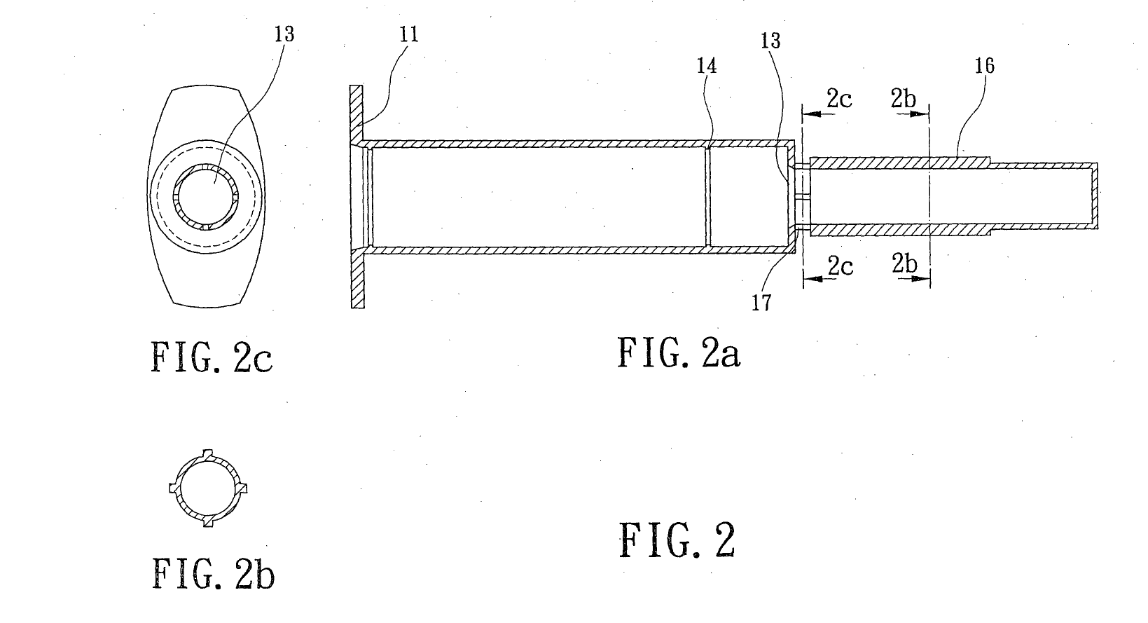

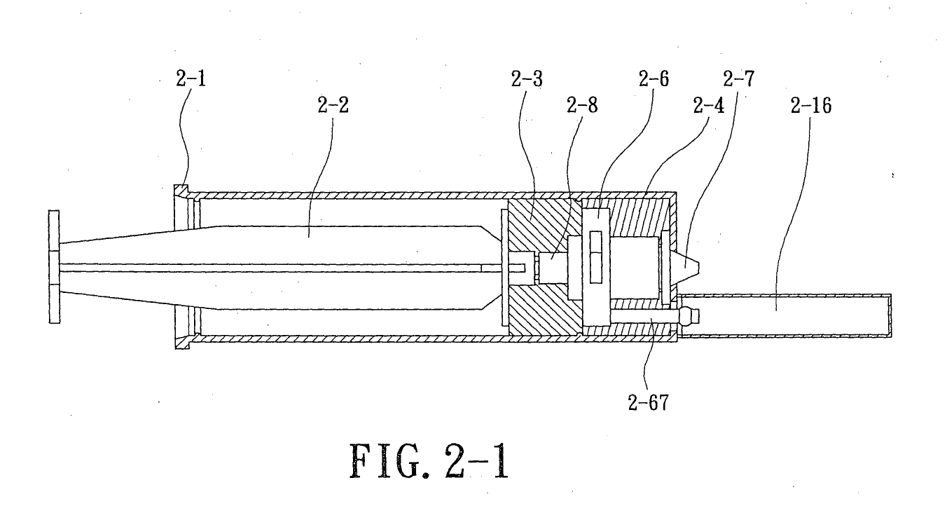

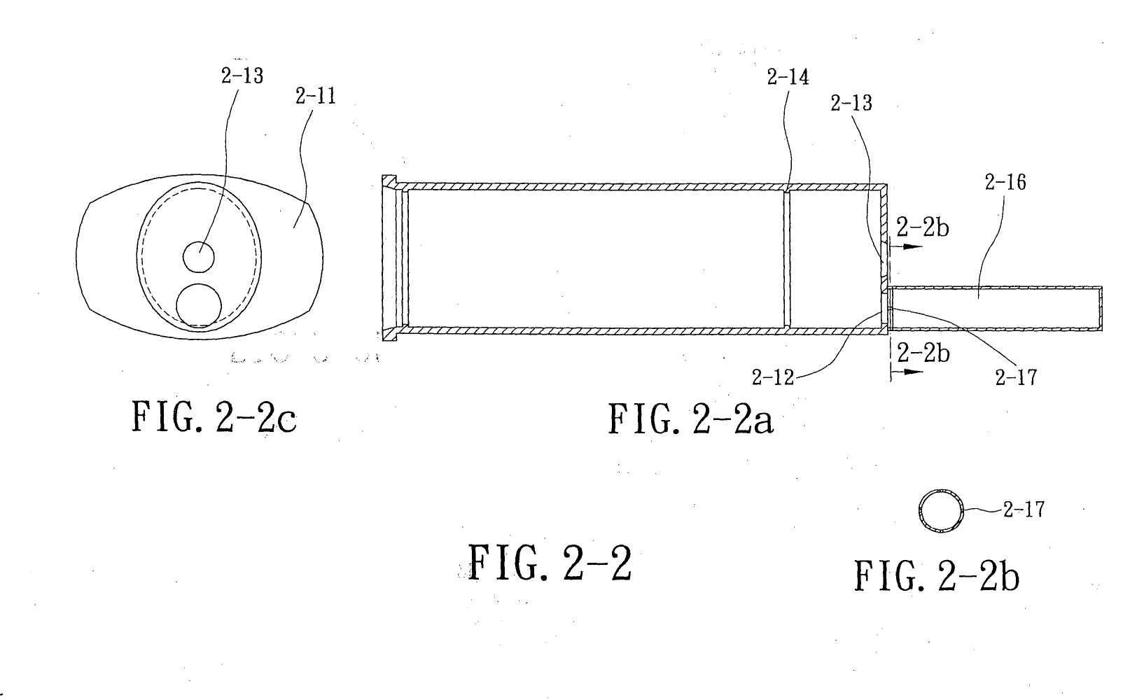

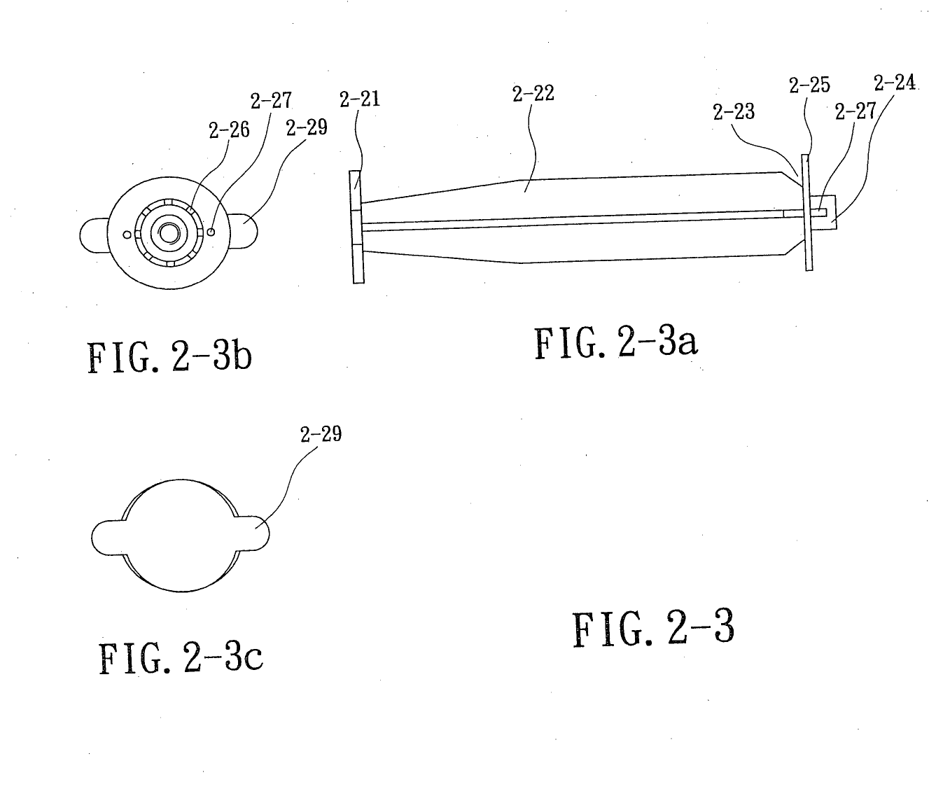

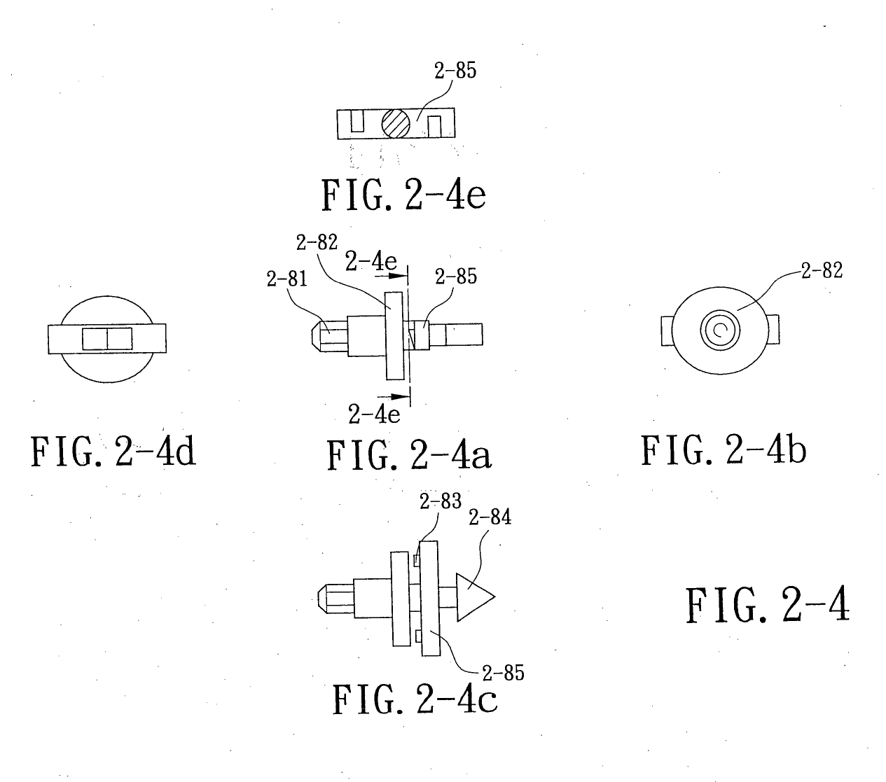

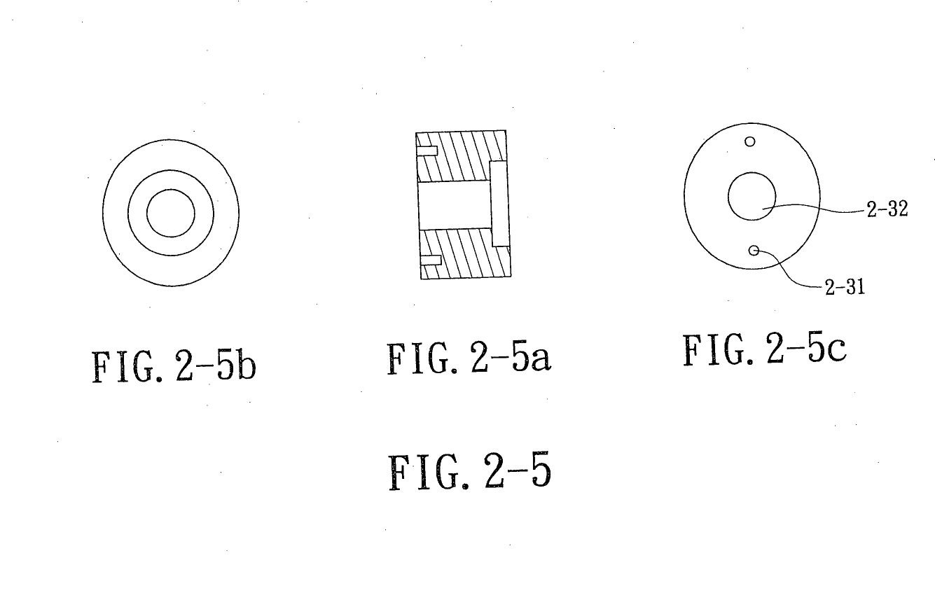

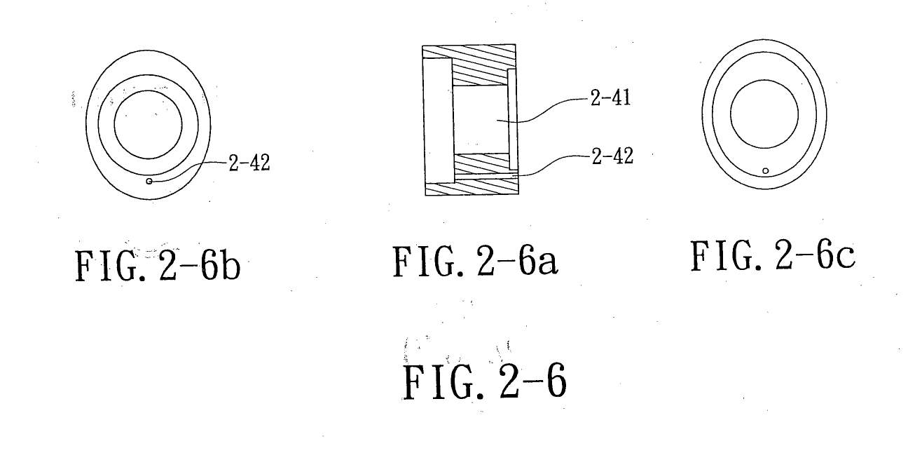

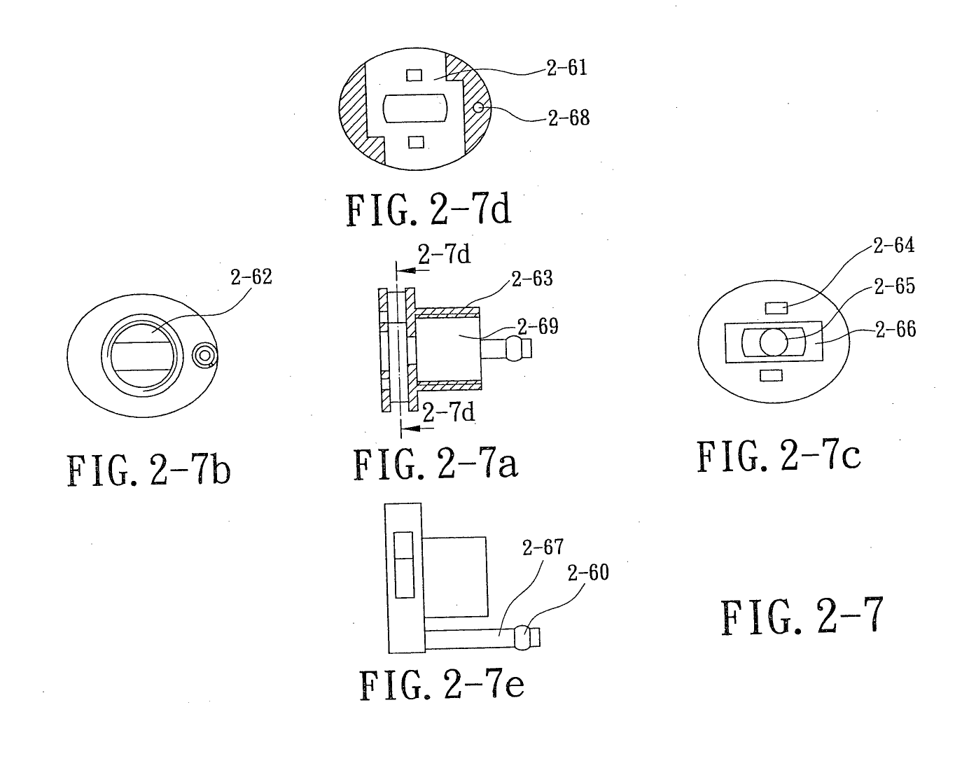

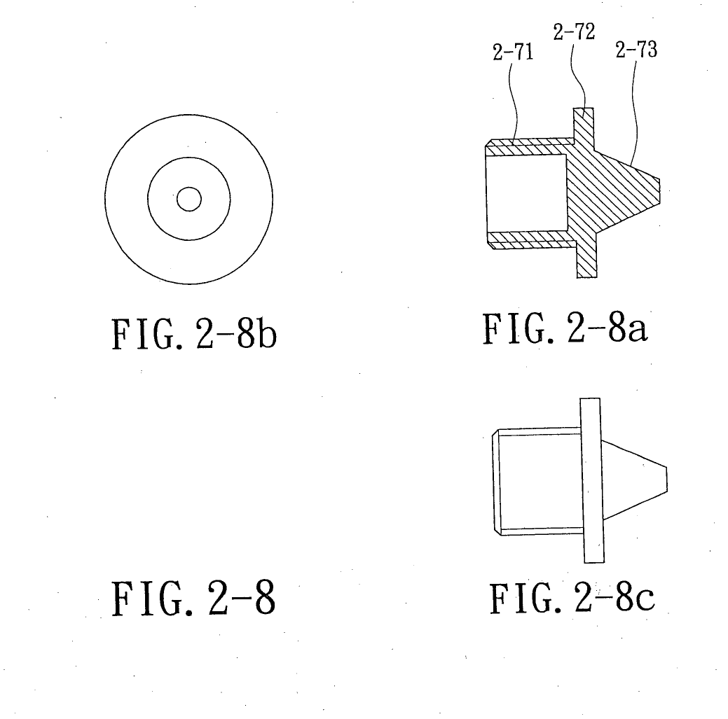

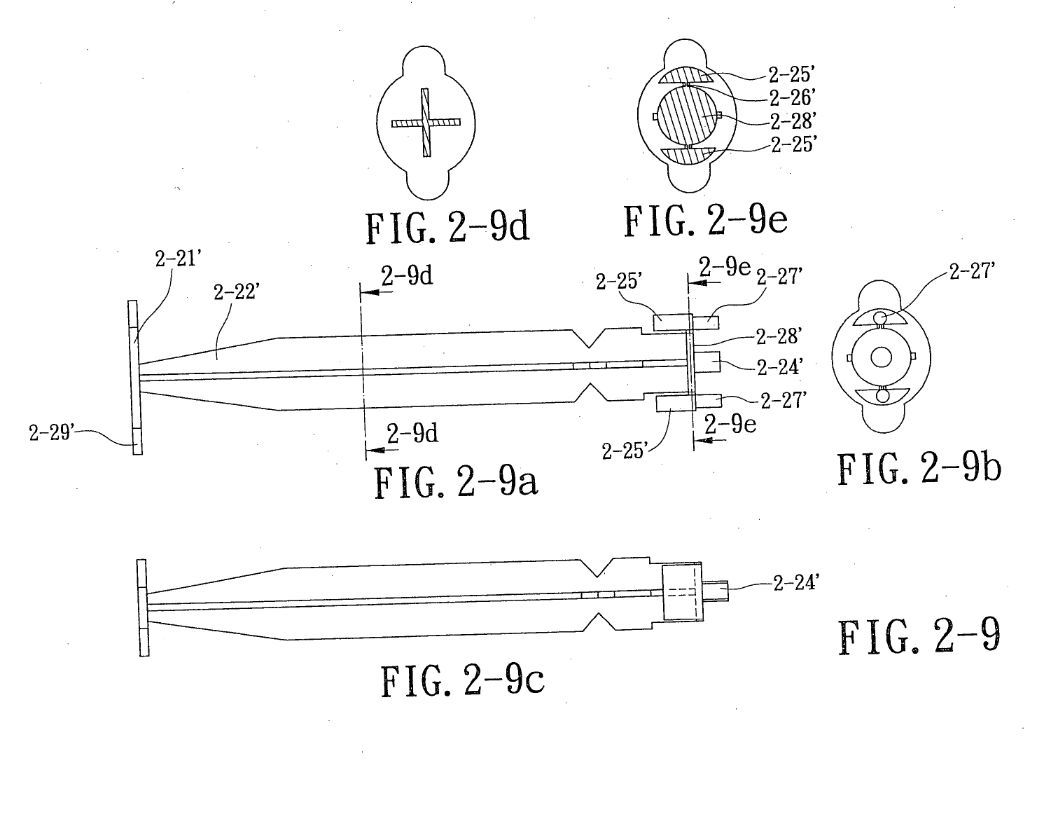

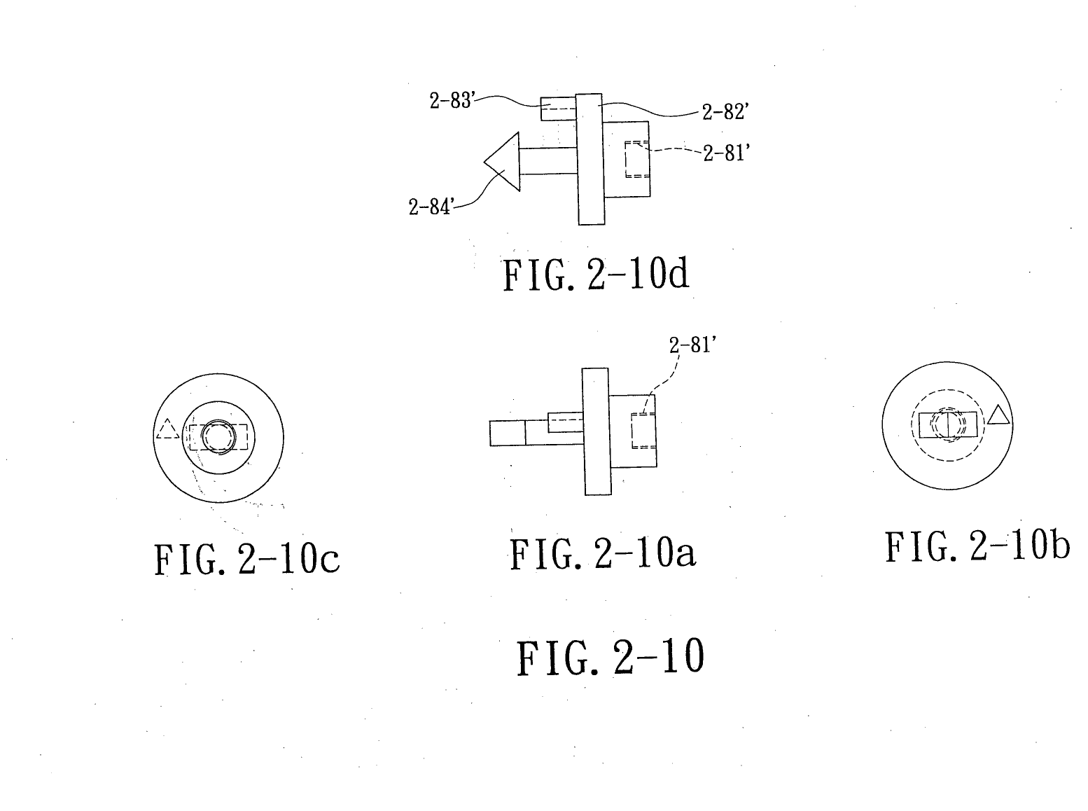

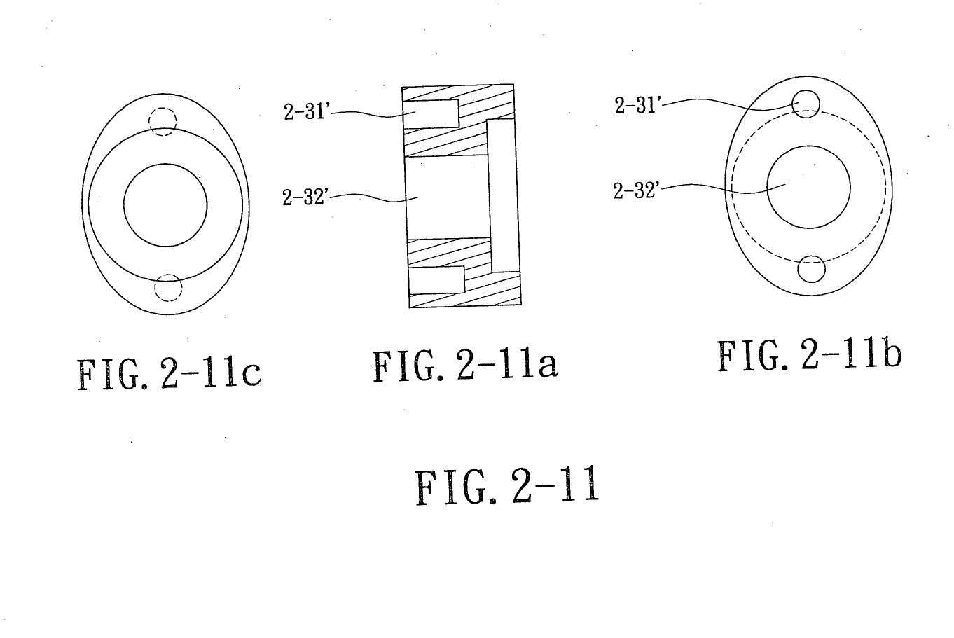

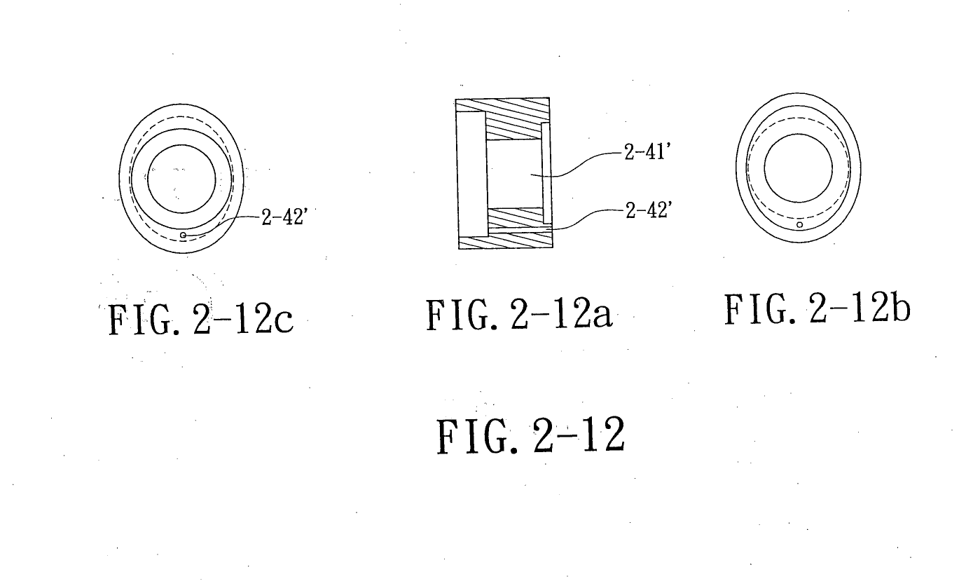

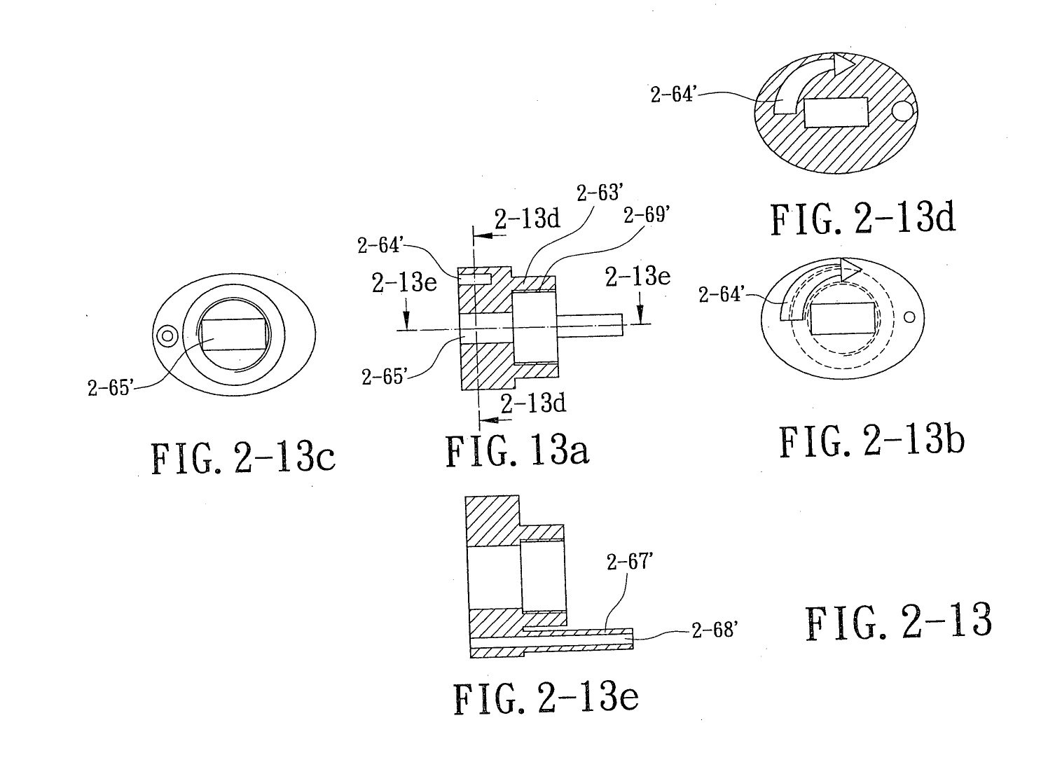

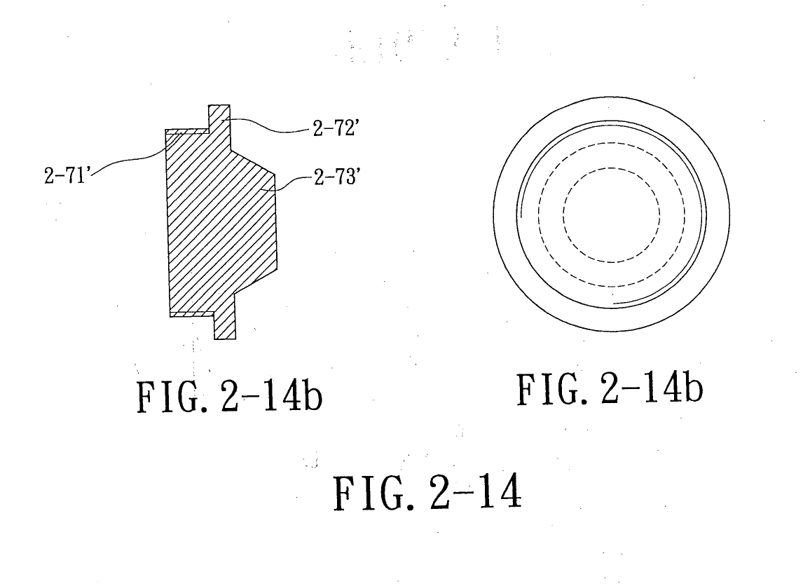

FIG. 1-2 shows structures of a needle cylinder and a needle cap of the present invention, wherein FIG. 1-2a is a main diagram, FIG. 1-2b is a perspective view along line B-B and FIG. 1-2c is a perspective view along line A-A;

FIG. 1-3 shows a structure of a needle plunger of the present invention, wherein FIG. 1-3a is a main diagram, FIG. 1-3b is a right side view, FIG. 1-3c is a left side view and FIG. 1-3d is a cross-sectional view along line A-A;

FIG. 1-4 shows a structure of a plunger hanger of the present invention, wherein FIG. 1-4a is a main diagram, FIG. 1-4b is a left side view, FIG. 1-4c is a top view, FIG. 1-4d is a cross-sectional view along line A-A and FIG. 1-4e is a right side view;

FIG. 1-5 shows a structure of a plunger piston of the present invention, wherein FIG. 1-5a is a main diagram, FIG. 1-5b is a top view and FIG. 1-5c is a bottom view;

FIG. 1-6 shows a structure of a needle holder piston of the present invention, wherein FIG. 1-6a is a main diagram, FIG. 1-6b is a right side view and FIG. 1-6c is a left side view;

FIG. 1-7 shows a structure of a hanger cavity of the present invention, wherein FIG. 1-7a is a main diagram, FIG. 1-7b is a right side view, FIG. 1-7c is a left side view, FIG. 1-7d is a perspective view along line A-A and FIG. 1-7e is a top view;

FIG. 1-8 shows a structure of a needle holder of the present invention, wherein FIG. 1-8a is a main diagram, FIG. 1-8b is a left side view and FIG. 1-8c is a top view;

FIG. 2-1 is a whole structure diagram according to a second embodiment of the present invention;

FIG. 2-2 shows structures of a needle cylinder and a needle cap of the present invention, wherein FIG. 2-2a is a main diagram, FIG. 2-2b is a perspective view along line B-B and FIG. 2-2c is a right side view;

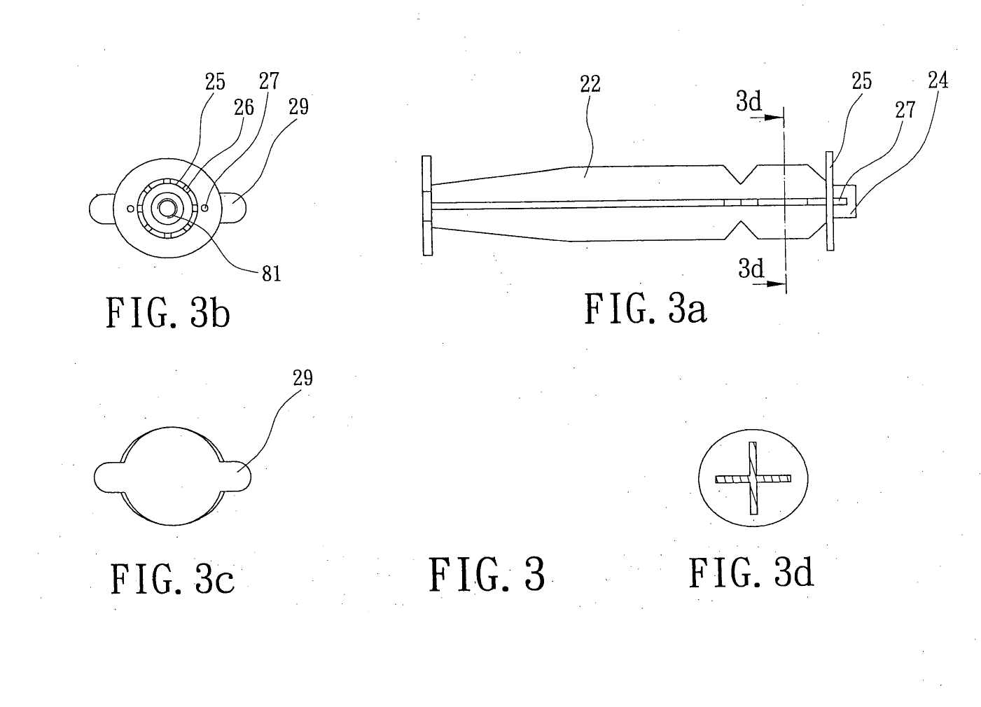

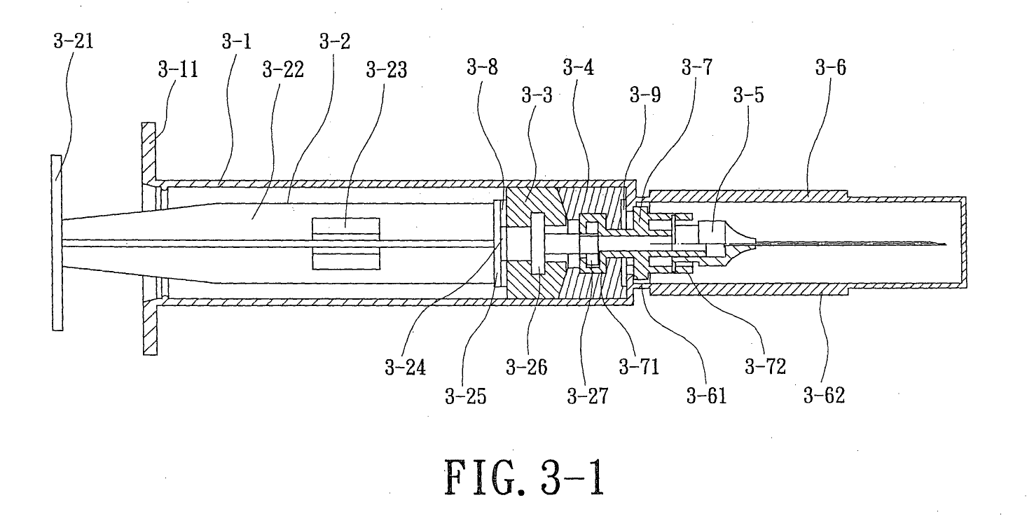

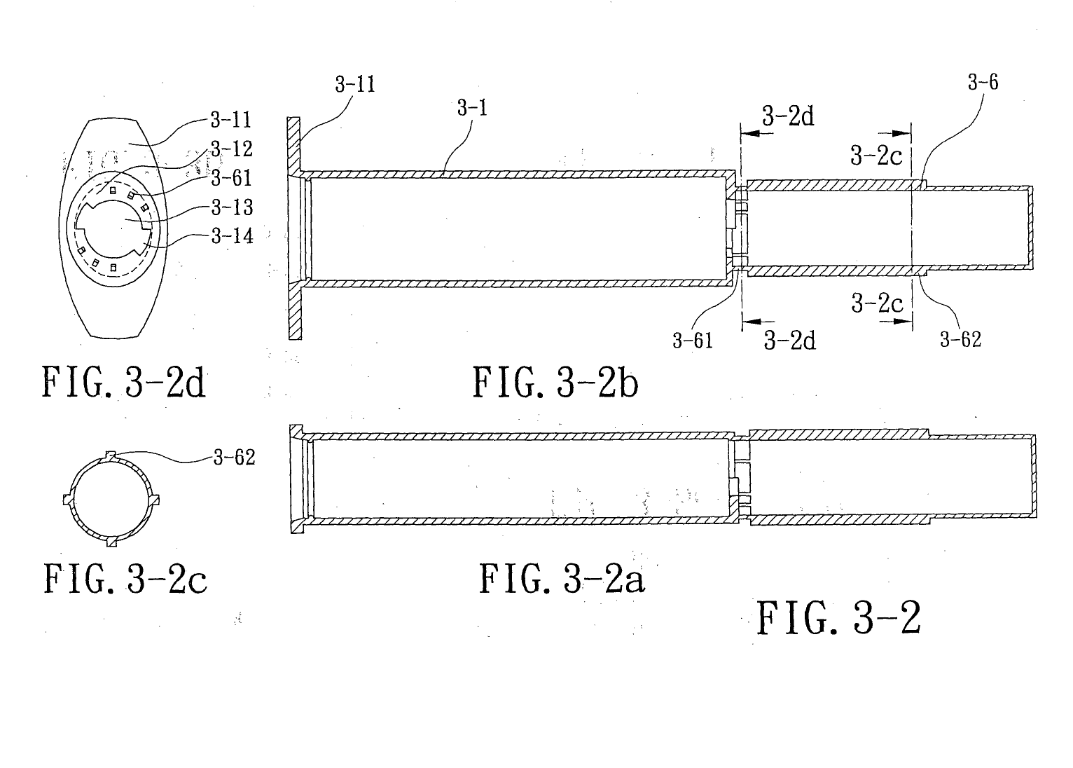

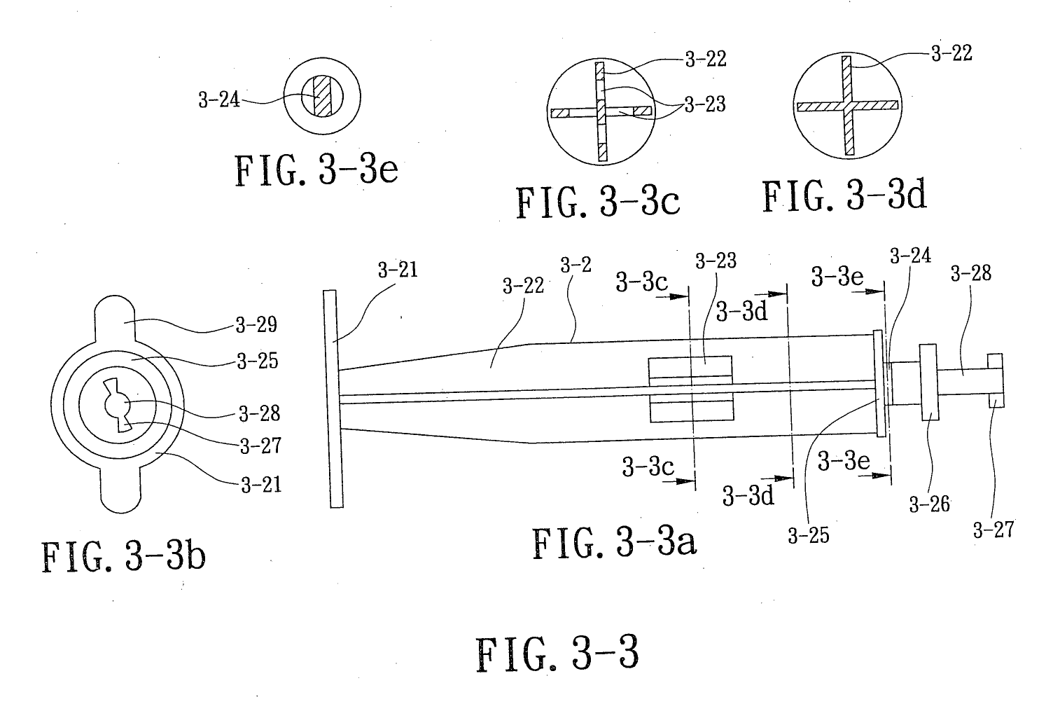

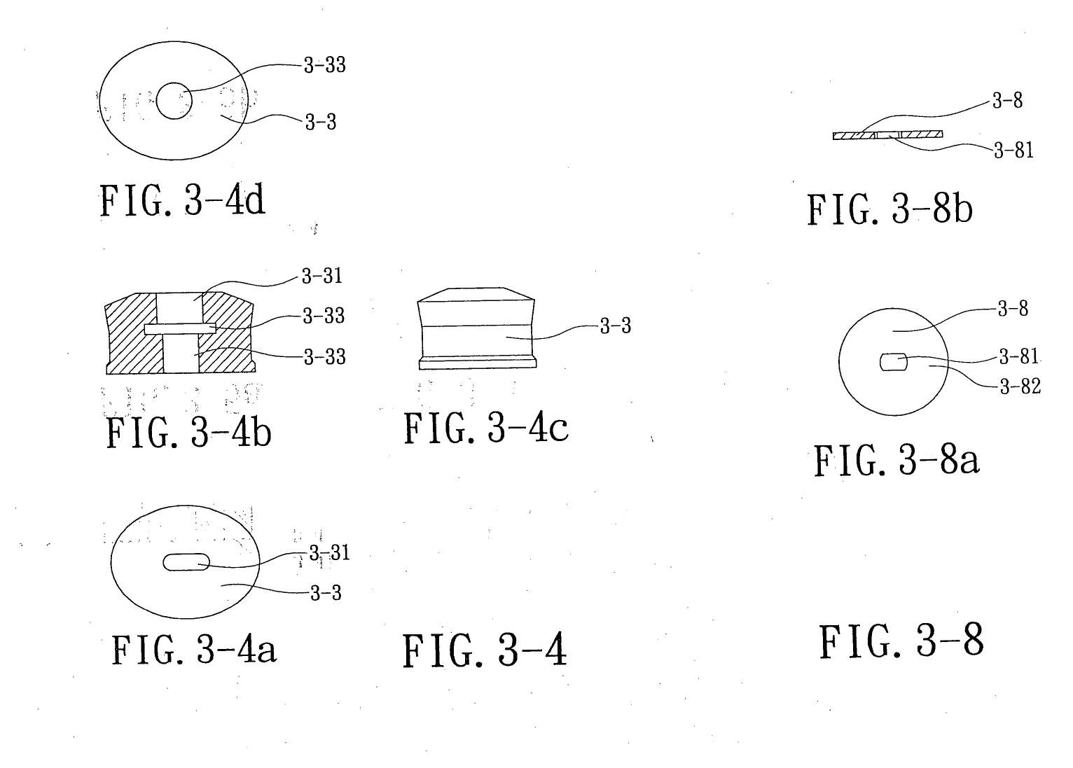

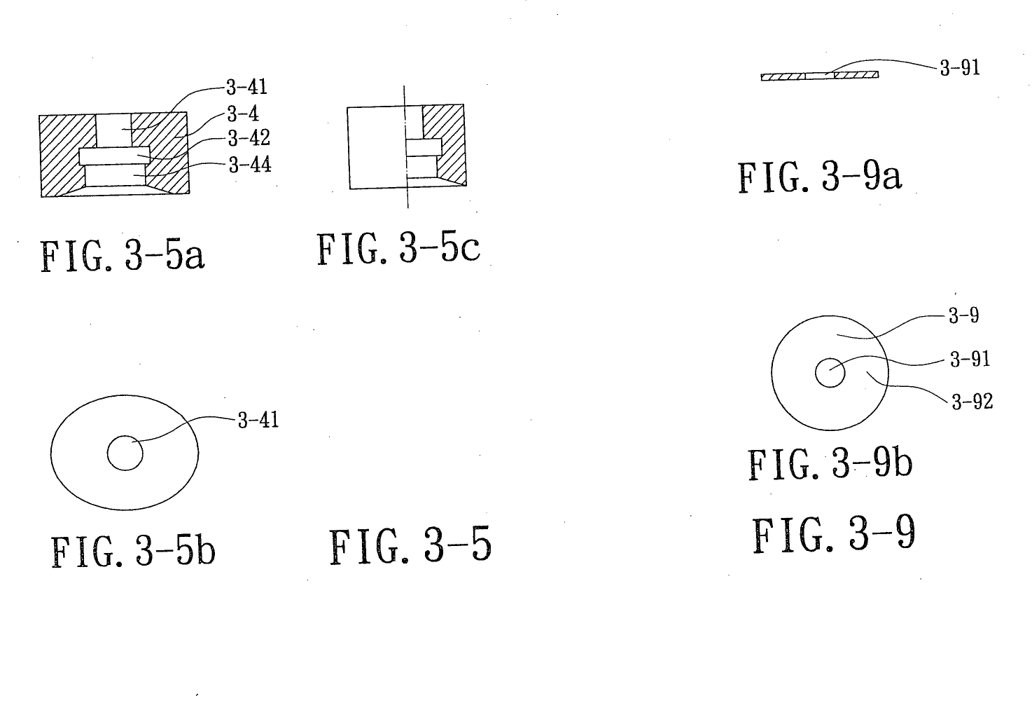

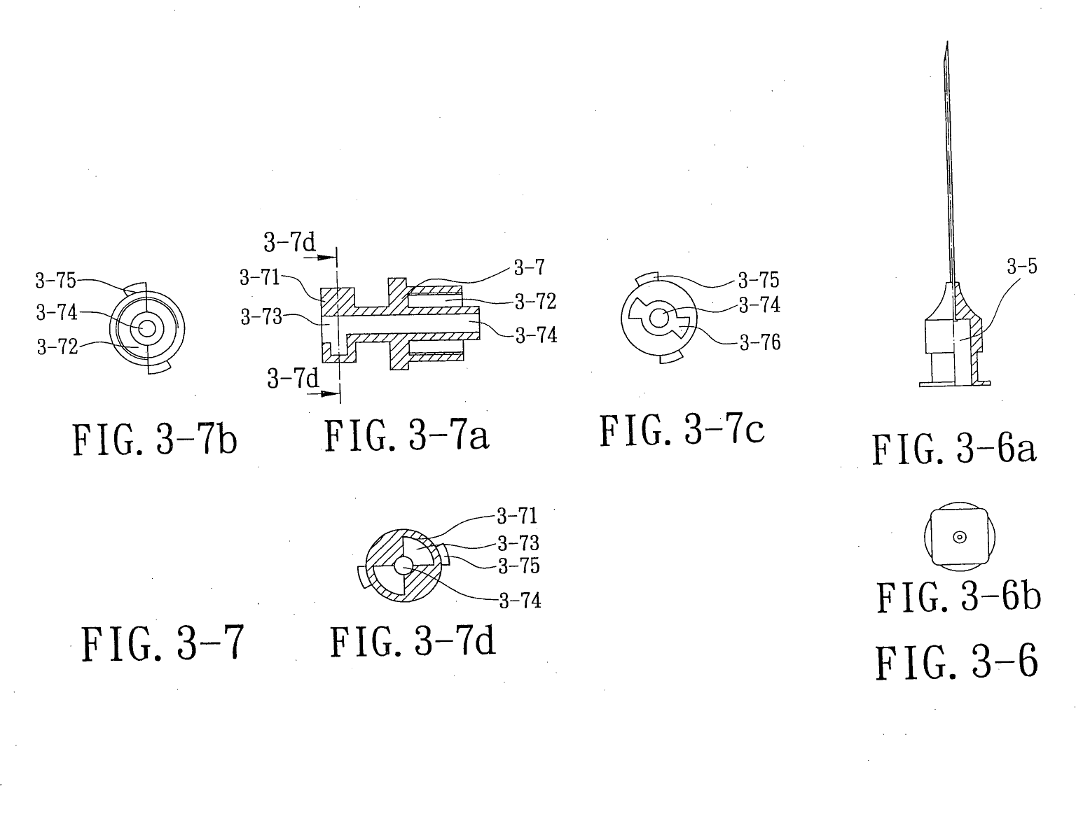

FIG. 2-3 shows a structure of a needle plunger of the present invention, wherein FIG. 2-3a is a main diagram, FIG. 2-3b is a right side view and FIG. 2-3c is a left side view;

FIG. 2-4 shows a structure of a plunger hanger of the present invention, wherein FIG. 2-4a is a main diagram, FIG. 2-4b is a left side view, FIG. 2-4c is a top view, FIG. 2-4d is a right side view and FIG. 2-4e is a cross-sectional view along line A-A;

FIG. 2-5 shows a structure of a plunger piston of the present invention, wherein FIG. 2-5a is a main diagram, FIG. 2-Sb is a top view and FIG. 2-5c is a bottom view;

FIG. 2-6 shows a structure of a needle holder piston of the present invention, wherein FIG. 2-6a is a main diagram, FIG. 2-6b is a right side view and FIG. 2-6c is a left side view;

FIG. 2-7 shows a structure of a hanger cavity of the present invention, wherein FIG. 2-7a is a main diagram, FIG. 2-7b is a right side view, FIG. 2-7c is a left side view, FIG. 2-7d is a perspective view along line A-A and FIG. 2-7e is a top view;

FIG. 2-8 shows a structure of a needle holder of the present invention, wherein FIG. 2-8a is a main diagram, FIG. 2-8b is a right side view and FIG. 2-8c is a top view;

FIG. 2-9 shows another structure of a needle plunger according to the second embodiment of the present invention, wherein FIG. 2-9a is a main diagram, FIG. 2-9b is a right side view, FIG. 2-9c is a top view, FIG. 2-9d is a cross-sectional view along line A-A and FIG. 2-9e is a cross-sectional view along line B-B;

FIG. 2-10 shows another structure of a plunger hanger of the present invention, wherein FIG. 2-10a is a main diagram, FIG. 2-10b is a left side view, FIG. 2-10c is a right side view and FIG. 2-10d is a bottom view;

FIG. 2-11 shows another structure of a plunger piston of the present invention, wherein FIG. 2-11a is a main diagram, FIG. 2-11b is a left side view and FIG. 2-11c is a right side view;

FIG. 2-12 shows another structure of a needle holder piston of the present invention, wherein FIG. 2-12a is a main diagram, FIG. 2-12b is a left side view and FIG. 2-12c is a right side view;

FIG. 2-13 shows another structure of a hanger cavity of the present invention, wherein FIG. 2-13a is a main diagram, FIG. 2-13b is a left side view, FIG. 2-13c is a right side view, FIG. 2-13d is a cross-sectional view along line A-A and FIG. 2-13e is a cross-sectional view along line B-B;

FIG. 2-14 shows another structure of a needle holder of the present invention, wherein FIG. 2-14a is a main diagram and FIG. 2-14b is a left side view;

FIG. 3-1 is a whole structure diagram according to a third embodiment of the present invention;

FIG. 3-2 shows structures of a needle cylinder and a needle cap of the present invention, wherein FIG. 3-2a is a top view, FIG. 3-2b is a main diagram, FIG. 3-2c is a left side view, FIG. 3-2d is a perspective view along line B-B and FIG. 3-2e is a perspective view along line A-A;

FIG. 3-3 shows a structure of a needle plunger of the present invention, wherein FIG. 3-3a is a main diagram, FIG. 3-3b is a right side view, FIG. 3-3c is a cross-sectional view along line C-C, FIG. 3-3d is a cross-sectional view along line B-B and FIG. 3-3e is a cross-sectional view along line A-A;

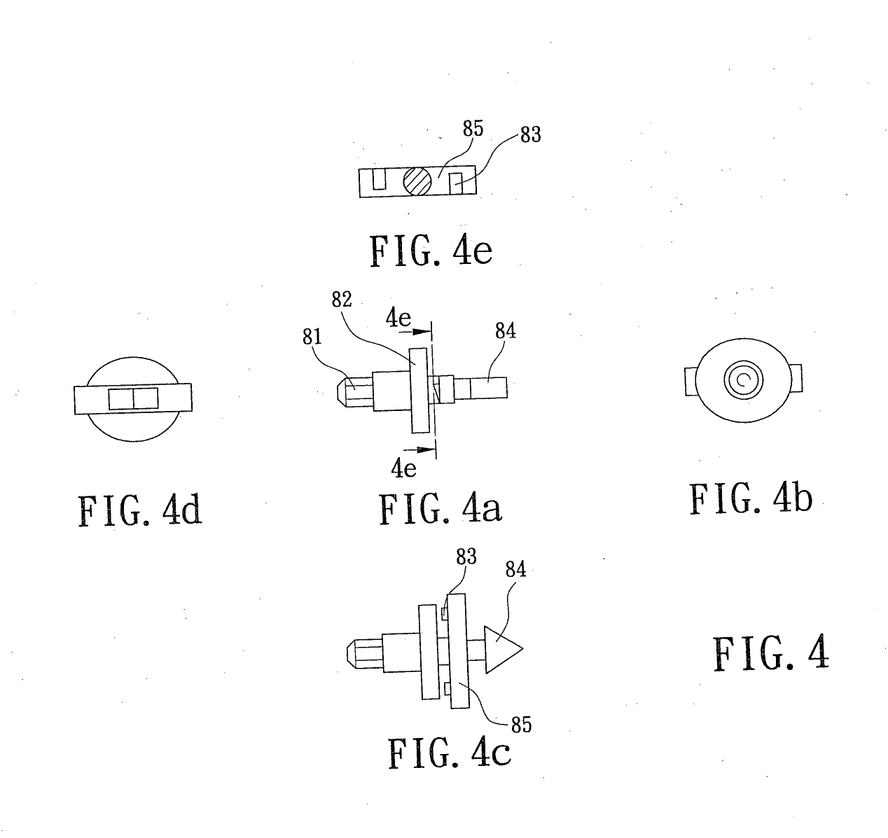

FIG. 3-4 shows a structure of a plunger piston of the present invention, wherein FIG. 3-4a is a top view, FIG. 3-4b is a main diagram, FIG. 3-4c is a left side view and FIG. 3-4d is a bottom view;

FIG. 3-5 shows a structure of a needle holder piston of the present invention, wherein FIG. 3-5a is a main diagram, FIG. 3-5b is a top view and FIG. 3-5c is a left side view;

FIG. 3-6 shows a structure of a needle of the present invention, wherein FIG. 3-6a is a main diagram and FIG. 3-6b is a top view;

FIG. 3-7 shows a structure of a needle holder of the present invention, wherein FIG. 3-7a is a main diagram, FIG. 3-7b is a right side view, FIG. 3-7c is a left side view and FIG. 3-7d is a perspective view along line A-A;

FIG. 3-8 shows a structure of a rear spacer of the present invention, wherein FIG. 3-8a is a top view and FIG. 3-8b is a main diagram; and

FIG. 3-9 shows a structure of a front spacer of the present invention, wherein FIG. 3-9a is a main diagram and FIG. 3-9b is a top view.

### DETAILED DESCRIPTION OF THE PREFERRED EMBODIMENTS

As shown in FIGS. 1 and 2, a syringe of the present invention comprises a needle cylinder 1, a plunger 2, a plunger hanger 8, a hanger cavity 6, a needle holder 7 and a needle 75. A handle 21 is provided at one end of the plunger 2. Symmetric protection wings 29 are provided at two ends of the handle 21. A plunger piston 3 is provided at the other end of the plunger 2. The handle 21 is engaged with the plunger piston 3 by a plunger body 22. The front end of the plunger piston 3 has a groove for receiving circular stop sheets of the hanger 8. There is a hole 13 at the bottom center of the needle cylinder 1, which is used to mount the needle holder 7. The edge of the hole 13 is of a coned loop shape. The needle holder 7 is mounted on the needle cylinder 1 by the piston 4 located on the bottom of the needle cylinder 1. A needle cap 16 and the needle cylinder 1 are incorporate, on which a weak destructing device 17 is provided. The destructing device 17 is destructed before use to accomplish the object of disposable use. The plunger body 22 has a breakable region. The breakable region is triangular breakable grooves provided at predetermined portions at the middle of the plunger body 22. The breakable region breaks after the syringe is used to accomplish the object of destructive protection.

As shown in FIGS. 3a to 3d, the cross-section of the plunger body 22 is a crossed structure. The breakable region on the plunger body 22 is triangular gaps on the four faces of the crossed structure at the middle of the plunger body 22. Other breakable regions can also be used according to the real situation. For instance, the breakable region on the plunger body 22 can be breakable gaps of other shapes at predetermined positions of the plunger body 22.

As shown in FIG. 3b, the destructing device on a piston holder 25 of the plunger body 22 is realized by providing a weak ring 26 on the elliptic piston holder 25.

As shown in FIGS. 3a and 3b, small columns 27 on the plunger body 22 are used to limit the position relationship between the plunger piston 3 and the plunger 2.

As shown in FIGS. 2a, 2b and 2c, the cross section of the needle cylinder 1 is elliptic. The end portion of the needle cylinder 1 has an expanded hand support 11. The front end of the inner wall of the needle cylinder 1 has ring-shaped protuberances for enhancing the firmness of a needle holder piston 4. The needle cap 16 and the needle cylinder 1 are incorporate, between which a weak destructing device 17 provided.

As shown in FIGS. 1, 3 and 4, the front end of the piston holder 25 of the plunger body 22 has a screw nut 24 engaged with the plunger hanger 8. The rear of the plunger hanger 8 has a thread 81 matched with the screw nut 24. The middle of the plunger hanger 8 has a projective circular disk 82 for fastening the plunger piston 3. The front end of the plunger hanger 8 has a rectangular pull hook 85 matched with the hanger cavity 6. The rectangular pull hook 85 has a triangular pillared fastener 83 having self-destructing function. The forefront end of the plunger hanger 8 is a triangular pull hook 84.

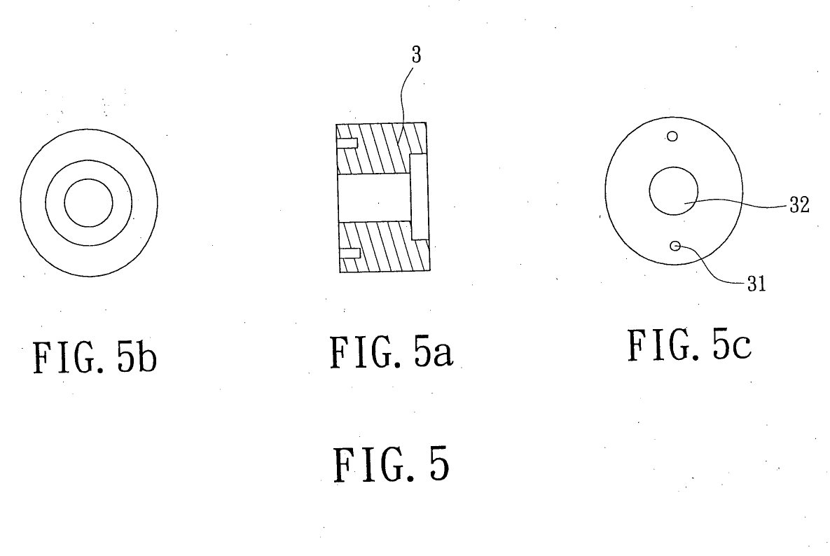

As shown in FIG. 5, the plunger piston 3 has a through hole 32 to be slipped into by the screw nut 24 at the front end of the plunger 2. The plunger piston 3 is of an elliptic cylindrical shape. The plunger piston 3 has a circular hole 31 matched with the small column 27 on the piston holder 25. Both the circular hole 31 and the plunger piston 3 can be made of thermoplastic, elastic material. The diameter of the through hole 32 is slightly smaller than that of the screw nut 24. External force can be exploited to slip the screw nut 24 into the through hole 32 to ensure firm installation of the plunger piston 3.

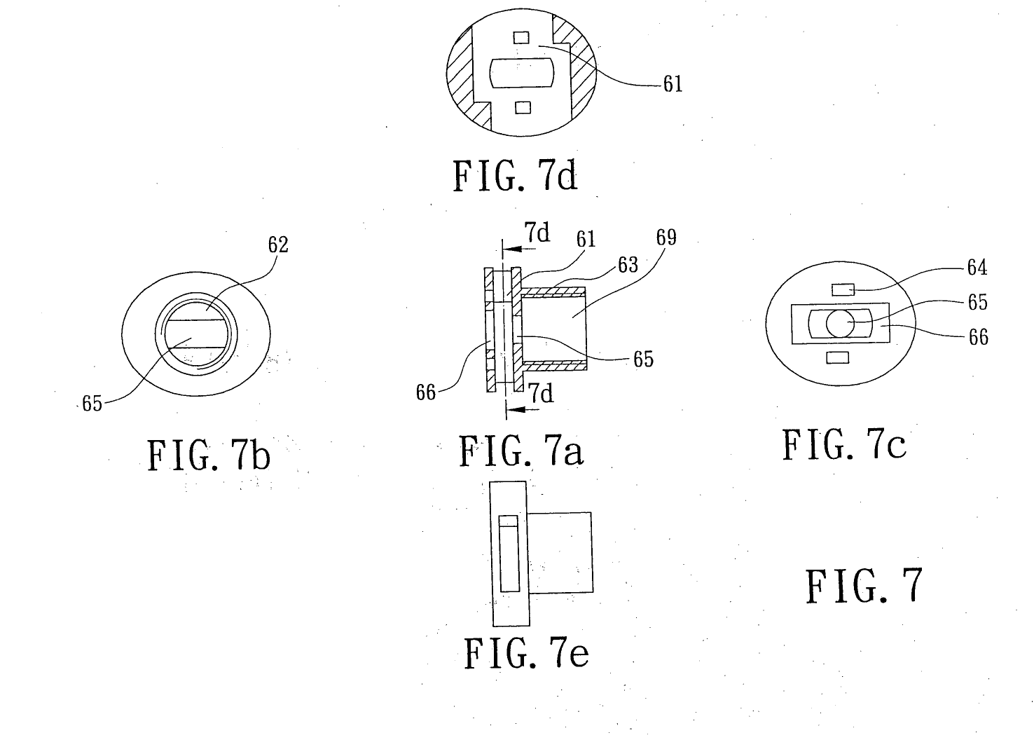

As show in FIG. 7, the bottom face of the hanger cavity 6 has a rectangular hole 66 similar to the rectangular pull hook 85. The rectangular hole 66 forms a cavity 61, which can let the rectangular pull hook 85 rotate a certain angle after sticking into the hanger cavity 6 a certain depth. The bottom face of the hanger cavity 6 has a rectangular hole 64 matched with the self-destructing fastener 83 on the rectangular pull hook 85. The front end of the cavity 61 are shrunk by two protection sheets 62 into a narrow slit 65, which can be passed through by the triangular pull hook 84 at the forefront of the plunger hanger 6. The narrow slit 65 is also the passageway of medicinal liquid. The front end of the hanger cavity 6 is a screw nut 63 matched with a thread 71 at the rear end of the needle holder 7.

As shown in FIG. 7, the screw nut 63 at the front end of the hanger cavity 6 has a hollow circular through hole 69, whose rear is shrunk by two protection sheets 62 into a narrow slit 65, which can be passed through by the triangular pull hook 84 at the forefront of the plunger hanger 6. The through hole 69 is a passageway of drug, and is also a cavity for rotation of the triangular pull hook 84.

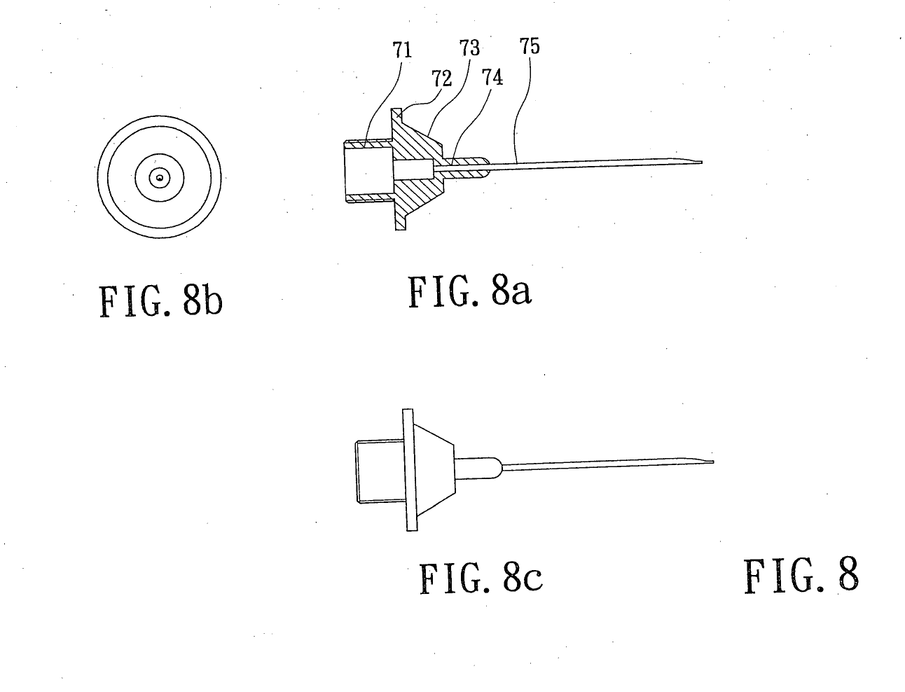

As shown in FIG. 8, the thread 71 at the rear end of the needle holder 7 is matched with the screw nut 63 at the front end of the hanger cavity 6. The rear end having a thread is a hollow circular through hole.

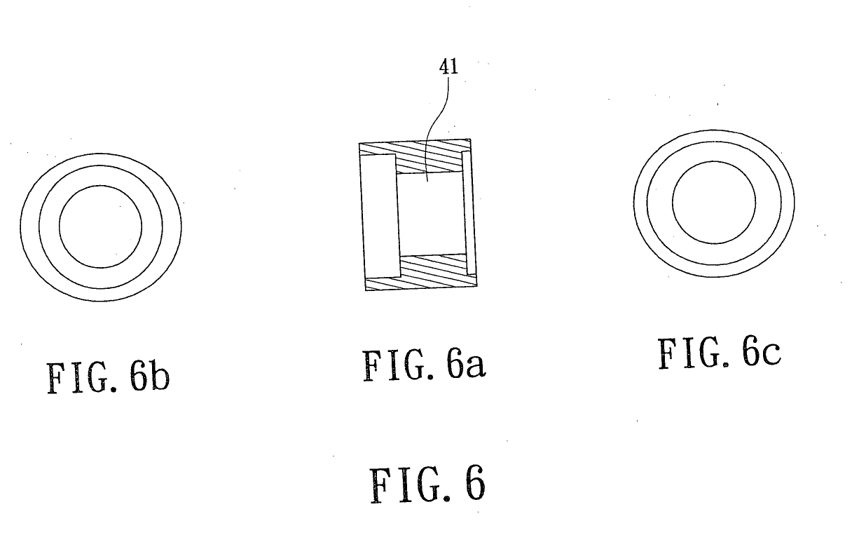

As shown in FIGS. 6 and 8, there is a through hole 74 at the center of the needle holder 67. The middle of the needle holder 7 has a coned needle lock 73. Please also refer to FIGS. 1 and 2. The mounting hole 13 at the center of the bottom of the needle cylinder 1 is a circular hole, which can be passed through by the coned needle lock 73 of the needle holder 7 with a slight force. The needle holder piston 4 is of an elliptic cylindrical shape, whose outer diameter is matched with the inner diameter of the needle cylinder 1. The center of the needle holder piston 4 has a needle holder hole 41, which can tightly mount the needle holder 7. In order to prevent the needle holder piston 4 from slipping off when pulled back, the needle holder 7 has a stop sheet 72. The front end of the needle holder 7 is connected with a needle 75 thereon.

As shown in FIGS. 1 and 2, the needle cap 16 and the needle cylinder 1 are incorporate, between which a weak destructing device 17 is provided. The end portion of the needle cap 16 has an opening matched with the circular hole 13 at the bottom of the needle cylinder 1. The needle cap 16 has a circular long hole for receiving the needle 75.

Back to FIG. 1, when the present invention is assembled, the needle holder piston 4 is slipped into the screw nut 63 of the hanger cavity 6, and the needle holder 7 is then screwed for assembling them three together. Because the needle holder piston 4 is made of thermoplastic, elastic material, their connection is tight. Next, the assembled part is slipped into the front portion of the needle cylinder from the rear of the needle cylinder 1 to let the coned needle lock 73 of the needle holder 7 protrude out of the bottom face of the needle cylinder 1 from the mounting hole 13 at the bottom of the needle cylinder 1 and be tightly fastened. The plunger piston 3 is slipped into the screw nut 24 at the front end of the plunger 2, and the plunger hanger 8 is then screwed for similarly assembling them three together. The assembled part is then slipped into the assembled needle cylinder part. When in use, the needle cap 16 and the needle cylinder 1 are first separated, the plunger 2 is then pulled out to suck in medicinal liquid for injection. The use method is the same as that of existent syringes. After injection is finished, because of positioning function of elliptic shape, the rectangular pull hook 85 of the plunger 2 will exactly align with the rectangular hole 66 of the hanger cavity 6 to let the pull hook 85 enter the cavity 61 of the hanger cavity 6 and the triangular pull hook 84 at the forefront end of the plunger hanger 8 enter the narrow slit 65 formed by the protection sheets 62 in the hanger cavity 6. The plunger 2 is then turned. Meanwhile, the weak ring of the plunger is destructed. The plunger hanger 8 also rotates a certain angle to let the self-destructing fastener 83 of the rectangular pull hook 85 enter the rectangular hole 64 at the bottom face of the hanger cavity 6 to form a self-destructing structure. Meanwhile, there is a certain angle between the triangular pull hook 84 and the narrow slit 65. Subsequently, the plunger 2 is pulled outwards. Meanwhile, the plunger 2, the plunger hanger 8, the needle holder piston 4, the hanger cavity 6, the needle holder 7 and the needle 75 are pulled into the needle cylinder 1 altogether. The plunger 2 is then broken with a force or the plunger is reversed to screw open the connection screw between the plunger 2 and the plunger hanger 8, hence retreating the plunger 2. Other parts are remained in the needle cylinder to accomplish the object of destructive protection.

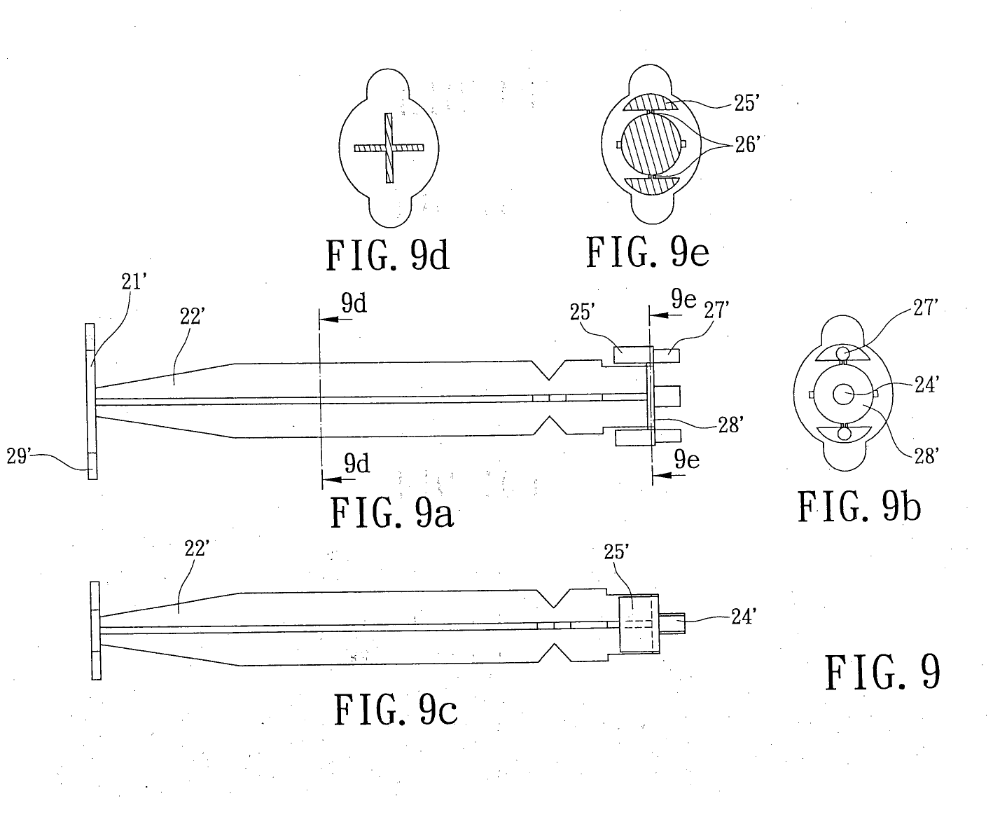

FIGS. 9 to 14 show a syringe according to another embodiment of the present invention.

As shown in FIG. 9, one end of a plunger 2′ has a handle 21′. Two ends of the handle 21 have symmetric protection wings 29′. The other end of the plunger 2′ has a circular plate 28′ transversally placed at the end portion thereof. Two symmetric semi-elliptic piston holders 25′ are provided at two edges of a crossed structure and outside the circular plate 28′. The profile of the two piston holders 25′ is an elliptic shape in accordance with the elliptic inner wall of the needle cylinder 1. The piston holders 25′ form point collection structures with the two edges of the crossed structure, hence forming destructing devices 26′ on the piston holders 25′. When the plunger is turned, the two connection points of the piston holders are destructed to accomplish self-destructing object. There is a screw rod 24′ engaged with the plunger hanger 8 is provided at the center of the front end of the circular plate 28′ of the plunger 2. Two small columns 27′ are symmetrically arranged on the two semi-elliptic piston holders 25′. The positions of the small columns 27′ correspond to those of circular holes 31′ on the rear end face of the plunger piston 3′. The small columns 27′ are used to limit the position relationship between the plunger piston 3′ and the plunger 2′.

Please refer to FIGS. 9a to 9e. The cross-section of the plunger body 22′ is a crossed structure. The breakable region on the plunger body 22′ is triangular gaps on the four faces of the crossed structure at the middle of the plunger body 22′. Other breakable regions can also be used according to the real situation. For instance, the breakable region on the plunger body 22′ can be breakable gaps of other shapes at predetermined positions of the plunger body 22′.

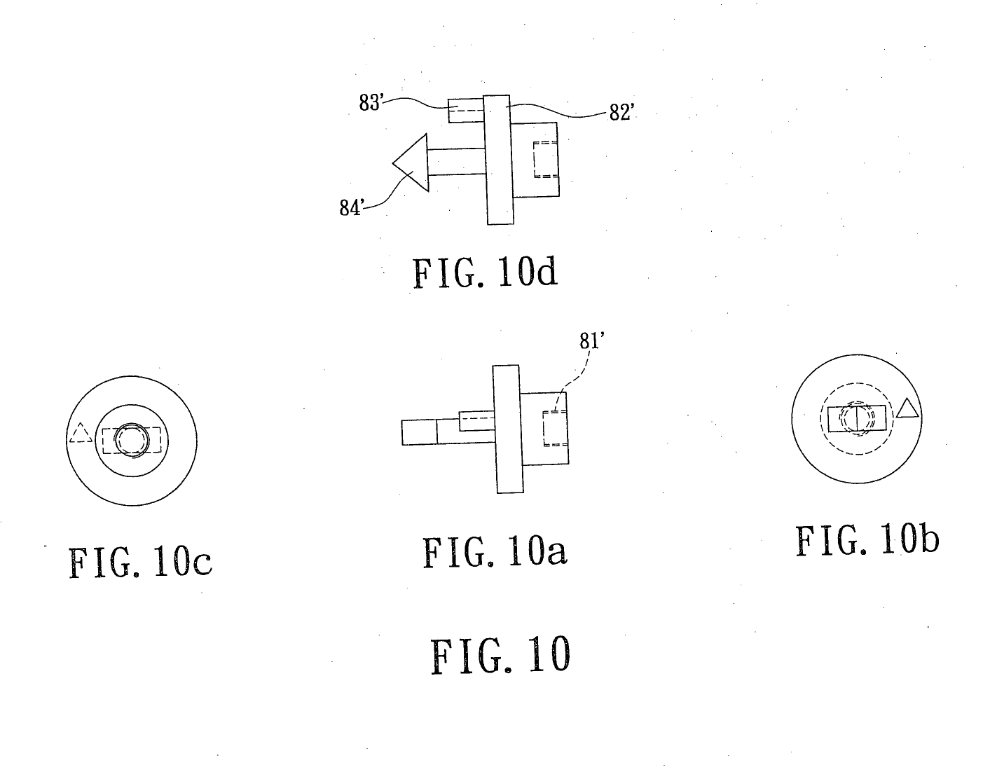

As shown in FIG. 10, the distal end of the plunger hanger 8′ has an inner thread 81′ connected with the plunger 2′. The middle of the plunger hanger 8′ has a projective circular disk 82′ for fastening the plunger piston 3′. The front end of the plunger hanger 8′ has an arrowheaded triangular pull hook 84′ matched with the hanger cavity 6′. A prismatic pillared self-destructing fastener 83′ parallel to the central line is provided at the outside of the front end face of the projective circular disk 82′ near the pull hook 84′.

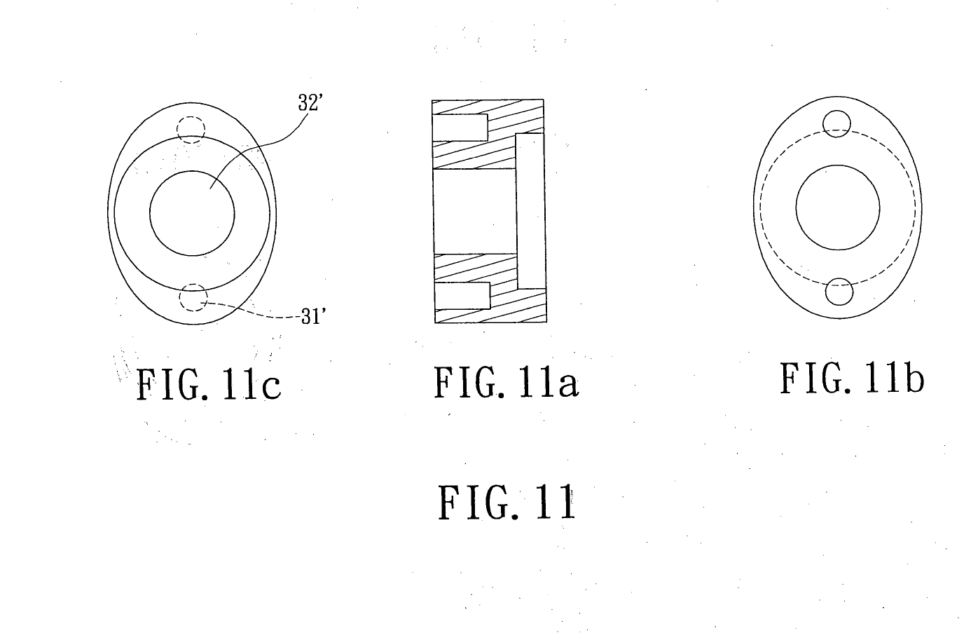

As shown in FIG. 11, the plunger piston 3′ has a through hole 32′, which can be slipped into by the outer circle at the rear end of the plunger hanger 8′. The plunger piston 3′ is elliptic. The plunger piston 3′ has a hole 31′ matched with a small column 27′ on a semicircular plunger piston holder 25′. Both the circular hole 31′ and the plunger piston 3′ can be made of thermoplastic, elastic material. The diameter of the through hole 32′ is slightly smaller than that of the outer circle at the rear end of the plunger hanger 8′. External force can be exploited to slip the outer circle at the rear end of the plunger hanger 8′ into the through hole 32′ to ensure firm installation of the plunger piston 3′.

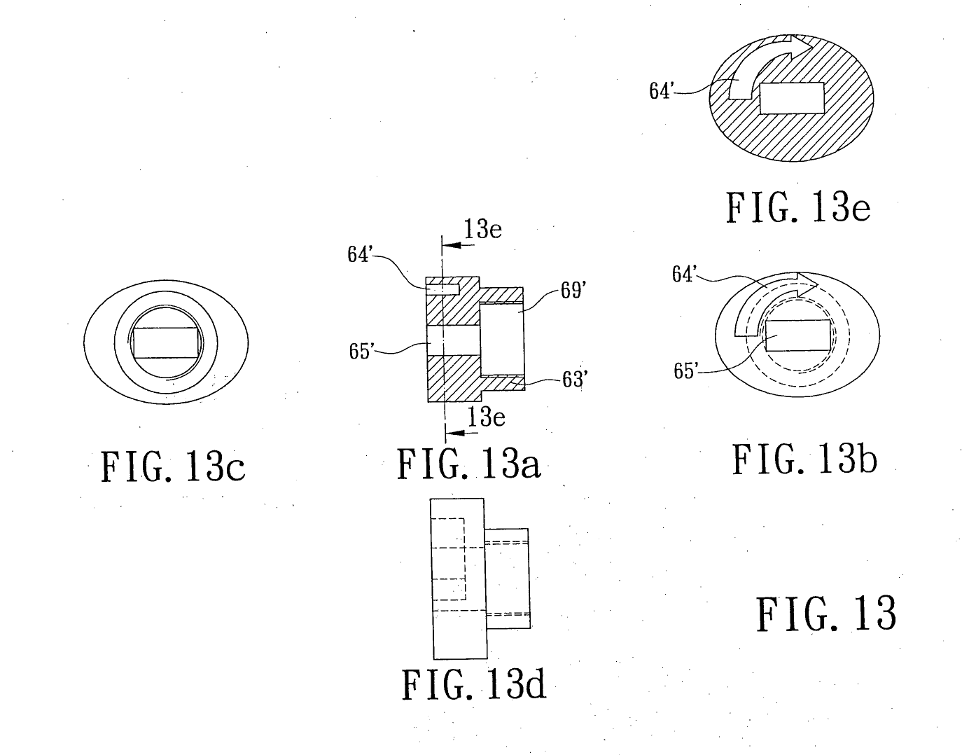

As shown in FIG. 13, the hanger cavity 6′ is elliptic, and has a rectangular hole 65′ with the same shape as the axial projection of the arrowheaded triangular pull hook 84′ at the center of one side near the pull hook. The long sides of the rectangular hole 65′ are exactly passed through by the triangular pull hook 84′, and the short sides thereof let the hook part of the triangular hook 84′ be retained and not able to recede. The rectangular hole 65′ can be passed through by the triangular pull hook 84′, and is also a passageway of medicinal liquid. The front end of the hanger cavity 6′ is a screw nut 63′ matched with a thread 71′ at the rear end of the needle holder 7′. An inner screw hole 69′ of the screw nut 63′ is a passageway of medicinal liquid, and is also a cavity for rotation of the triangular pull hook 84′. The one side of the hanger cavity 6′ near the pull hook has an annular pillar-shaped arrowheaded groove 64′, which is about a quarter circle from the horizontal to the vertical direction. The shape of the axial projection of the annular pillar-shaped groove 64′ is clockwise arrowheaded. The width of the annular pillar-shaped groove 64′ gradually diminishes from the start end to the distal end thereof, and ends with a triangular arrowhead at the distal end. As shown in FIG. 10, the prismatic pillared self-destructing fastener 83′ of the plunger hanger 8′ matches with the arrowheaded annular pillar-shaped groove 64′ of the hanger cavity 6′. The plunger hanger 8′ is turned to let the prismatic self-destructing fastener 83′ slide from the start end of the arrowheaded annular pillar-shaped groove 64′ into the arrowhead of the diminishing arrowheaded groove, be stuck in the arrowhead and be not able to recede, hence accomplishing self-destructing object.

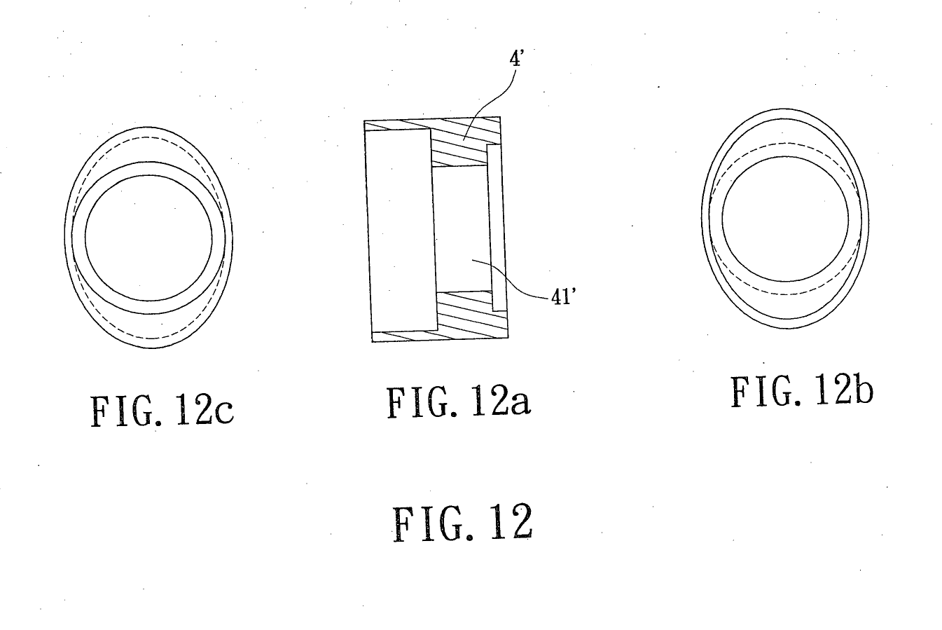

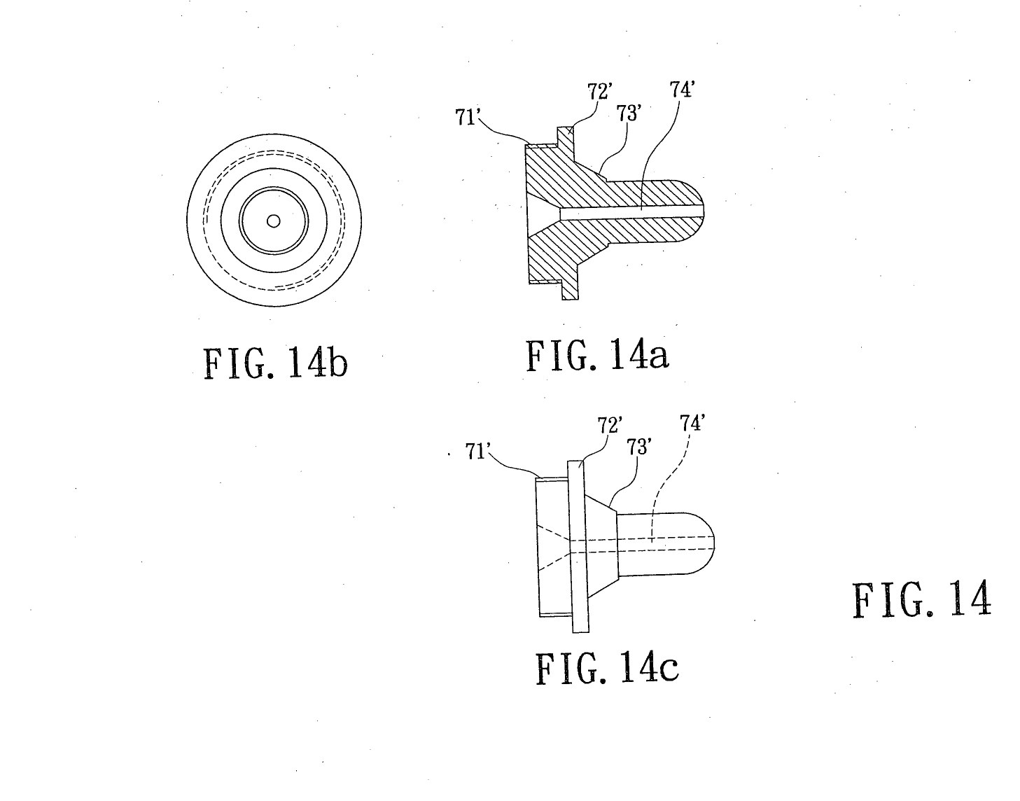

As shown in FIG. 14, an outer thread 71′ at the rear end of the needle holder 7′ matches with the screw nut 63′ at the front end of the hanger cavity 6′. The rear end having a thread is a hollow coned through hole. There is a through hole 74′ at the center of the needle holder 7′. The middle of the needle holder 7′ has a coned needle lock 73′. As shown in FIG. 12, the needle holder piston 4′ is of an elliptic cylindrical shape, whose outer diameter is matched with the inner diameter of the needle cylinder 1′. The center of the needle holder piston 4′ has a needle-holder hole 41′, which can tightly mount the needle holder 7′. In order to prevent the needle holder piston 4′ from slipping off when pulled back, the needle holder 7′ has a stop sheet 72′.

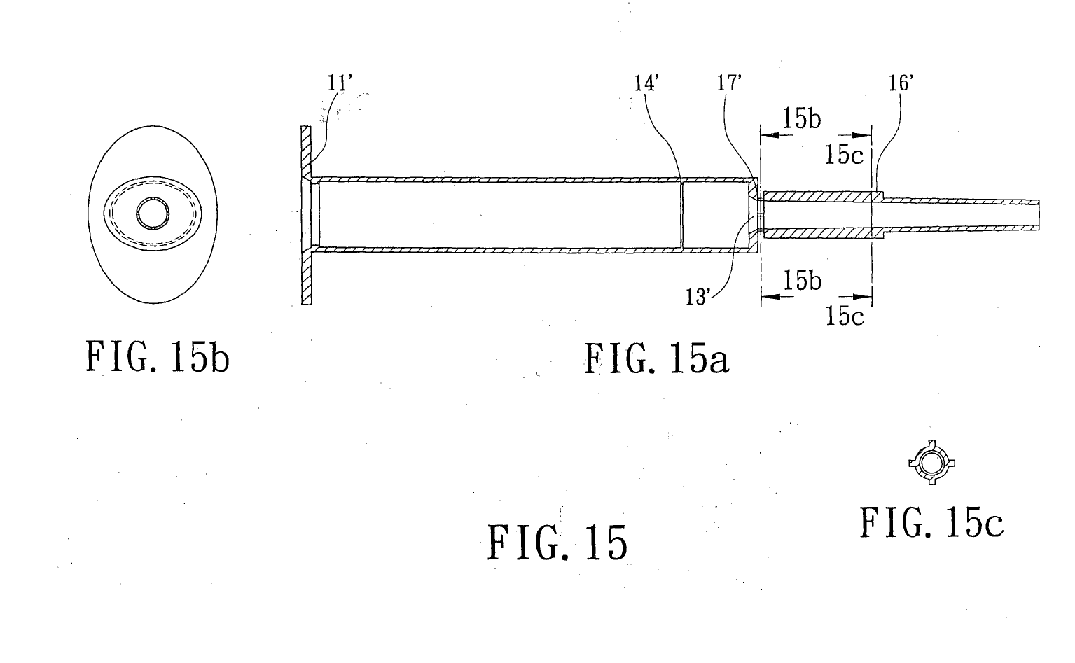

When this embodiment of the present invention is assembled, the needle holder (FIG. 14) first passes through the needle holder piston (FIG. 12) to be tightly assembled with the hanger cavity (FIG. 13) using the thread, is installed at the bottom of the needle cylinder from the rear opening of the needle cylinder 1′ (FIG. 15). The needle holder matches with the mounting hole at the center of the needle cylinder. A stainless steel needle body can be mounted at the front end of the needle holder when produced. Next, after the plunger (FIG. 9) is assembled with the plunger piston (FIG. 11), the small column of the plunger matches with the small cylindrical hole of the plunger piston, is tightly engaged with the plunger hanger (FIG. 10) by the thread, and is installed from the rear opening of the needle cylinder until tightly contacting with part of the hanger cavity. When in use, the needle cylinder and the needle cap are first separated (destructive permanent separation), the medicinal liquid is extracted and then injected, After injection is finished, the prismatic pillared fastener 83′ of the plunger hanger tightly contact the arrowheaded groove 64′ of the hanger cavity, the triangular pillared pull hook 84′ on the pull hook 8′ exactly passes through the rectangular hole 65′ in the hanger cavity. The plunger is then turned 90 degrees clockwise (the breakable point on the plunger is destructed). Meanwhile, the triangular pillared pull hook 84′ on the plunger hanger exactly hangs the two long sides of the rectangular hole 65′ of the hanger cavity 6′. At the same time, the prismatic pillared fastener 83′ on the plunger hanger passes through the arrowheaded groove 64′ gradually diminishing along with the rotation locus and forcibly enters the end portion of the arrowhead, hence letting the prismatic pillar be not able to recede. Accordingly, the triangular pillared pull hook 84′ on the plunger hanger cannot rotate, hence accomplishing self-destructing function. The plunger is then pulled back, and the needle head, needle holder, needle piston, and the hanger cavity are pulled into the needle cylinder altogether to ensure that the needle cylinder won't accidentally injure operators or recovery personnel. Finally, after the plunger is pulled back, it is turned counterclockwise to unfasten the thread for engagement of the plunger and the plunger hanger so as to decrease the amount of medical wastes.

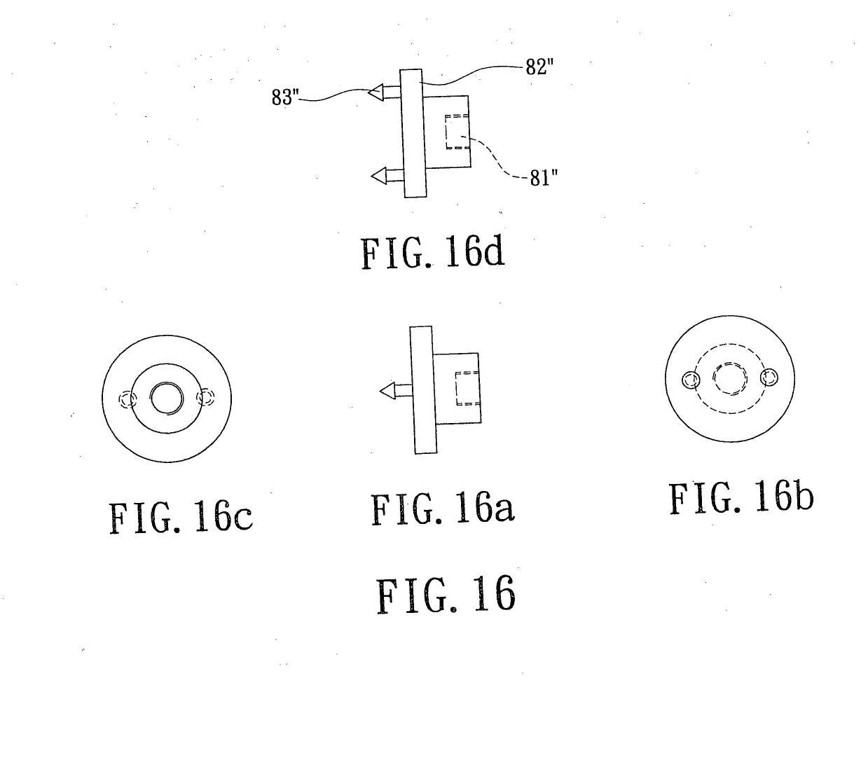

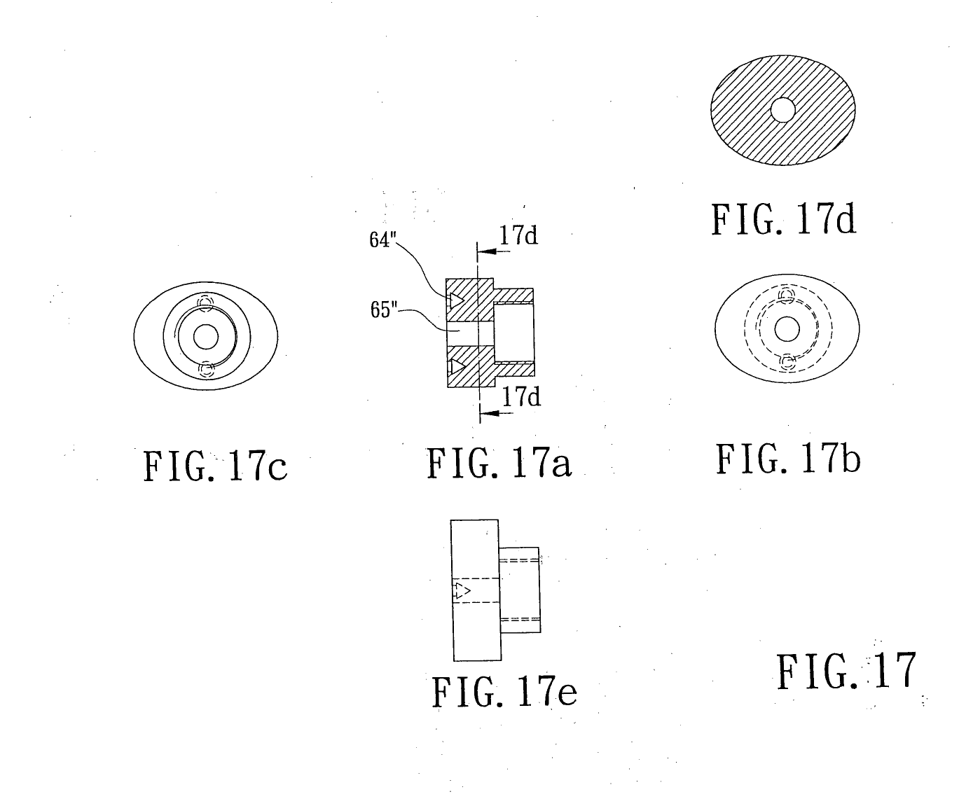

As shown in FIG. 16, the distal end of the plunger hanger 8″ has an inner thread 81″ engaged with the plunger 2″. The middle of the plunger hanger 8″ has a projective circular disk 82″ for fastening the plunger piston 3. The front end of the plunger hanger 8″ has two coned pull hooks 83″ matched with the hanger cavity 6″. As shown in FIG. 17, the hanger cavity 6″ is elliptic, and has a hole 65″ at the center of one side near the pull hook. The hole 65′ is a passageway of medicinal liquid. The side face of the hanger cavity 6″ has also two coned holes 64″ matched with the coned pull hooks 83″ of the plunger hanger 8″. The outer end of the coned hole is a pillared hole smaller than the maximum outer diameter of the coned pull hook 83″, and the inner end thereof is a cone having the same shape as the coned pull hook 83″. After the medicinal liquid is injected, the coned pull hooks 83″ of the plunger hanger are exactly inserted into the coned holes 64″ and cannot recede, hence accomplishing self-destructing object.

Care must be taken when adopting this kind of the plunger hanger 8″ and the hanger cavity 6″. After the plunger (FIG. 9) is assembled with the plunger piston (FIG. 11), the small column of the plunger correspondingly matches the small cylindrical hole of the plunger piston. Next, after the plunger is tightly engaged with the plunger hanger 8″ (FIG. 16) by the thread, it is installed from the rear opening of the needle cylinder until contacting part gap of the hanger cavity 6″. The plunger shouldn't be forcibly pulled into the small circular hole of the hanger cavity to avoid activation of the self-destructing device and waste of the syringe. When in use, after injection is finished, the triangular pillared pull hook 83″ of the hanger is forcibly inserted into the triangular coned hole 64″ of the hanger cavity to perform forcible deadlock, hence accomplishing self-destructing function.

As shown in FIG. 1-1, a syringe of the present invention comprises a needle cylinder 1-1, a plunger 1-2, a plunger hanger 1-8, a hanger cavity 1-6 and a needle holder 1-7. A handle 1-21 is provided on one end of the plunger 1-2. The handle 1-21 has symmetric protection wings 1-29. The other end of the plunger 1-2 has a plunger piston 1-3. The handle 1-21 is engaged with the plunger piston 1-3 by a plunger body 1-22. There is a hole 1-13 on the center from bottom of the needle cylinder 1-1, which is used to mount the needle holder 1-7. The needle holder 1-7 is mounted on the needle cylinder 1-1 by the plunger piston 1-4 located on the bottom of the needle cylinder 1-1. A needle cap 1-16 and the needle cylinder 1-1 are incorporate, on which a weak destructing device 1-17 is provided. The destructing device 1-17 is destructed before use to accomplish the object of disposable use. The plunger body 1-22 has a breakable region. The breakable region is opening at the middle of the plunger body 1-22 or triangular breakable grooves at predetermined portions of the plunger body 1-22. The breakable region breaks after the syringe is used to accomplish the object of destructive protection.

As shown in FIGS. 1-3a, 1-3c and 1-3d, the cross-section of the plunger body 1-22 is a crossed structure. Openings 1-23 of the breakable region on the plunger body 1-22 are on the four faces of the crossed structure at the middle the plunger body 1-22. Other breakable regions can also be-used according to the real situation. For instance, the breakable region on the plunger body 1-22 can be breakable gaps of other shapes at predetermined positions of the plunger body 1-22.

The opening 1-23 is triangular, whose area is 30% to 80% of the area of the cross section of the plunger body 1-22.

As shown in FIG. 1-3b, the destructing device on the piston holder 1-25 of the plunger body 1-22 is embodied by providing a weak ring 1-26 on the elliptic piston holder 1-25. A triangular gap 1-20 of the plunger body 1-22 is provided at the connection position of the four faces of the crossed structure of the plunger body 1-22 and the piston holder 1-25. The gap 1-20 is connected with the inner circle of the destructing device 1-26. The gap 1-20 can also be of any shape like rectangular.

As shown in FIGS. 1-3a and 1-3b, small columns 1-27 on the plunger body 1-22 are used to limit the position relationship between the plunger piston 1-3 and the plunger 1-2.

As shown in FIGS. 1-2a, 1-2b and 1-2c, the cross section of the needle cylinder 1-1 is elliptic. The end portion of the needle cylinder 1-1 has an expanded hand support 1-11. The front portion of the inner wall of the needle cylinder 1-1 has ring-shaped protuberances for enhancing the firmness of a needle holder piston 1-4. The needle cap 1-16 and the needle cylinder 1-1 are incorporate, between which a weak destructing device 1-17 provided.

As shown in FIGS. 1-1, 1-3 and 1-4, the front end of the piston holder 1-25 of the plunger body 1-22 has a screw nut 1-24 engaged with the plunger hanger 1-8. The rear of the plunger hanger 1-8 has a thread 1-81 matched with the screw nut 1-24. The middle of the plunger hanger 1-8 has a projective circular disk 1-82 for fastening the plunger piston 1-3. The front end of the plunger hanger 1-8 has a rectangular pull hook 1-85 matched with the hanger cavity 1-6. The rectangular pull hook 1-85 has a triangular pillared fastener 1-83 having self-destructing function. The forefront end of the plunger hanger 1-8 is a triangular pull hook 1-84.

As shown in FIG. 1-5, the plunger piston 1-3 has a through hole 1-32, which can be slipped into by the screw nut 1-24 at the front end of the plunger 1-2. The plunger piston 1-3 is of an elliptic cylindrical shape. The plunger piston 1-3 has a circular hole 1-31 matched with the small column 1-27 on the piston holder 1-25. The plunger piston 1-3 can be made of thermoplastic, elastic material. The diameter of the through hole 1-32 is slightly smaller than that of the screw nut 1-24. External force can be exploited to slip the screw nut 1-24 into the through hole 1-32 to ensure firm installation of the plunger piston 1-3 with the plunger 1-2.

As show in FIG. 1-7, the surface of the hanger cavity 1-6 has a rectangular hole 1-66 similar to the rectangular pull hook 1-85. The rectangular hole 1-66 sticks into the hanger cavity 1-6 a certain depth to form a cavity 1-61, which can let the rectangular pull hook 1-85 rotate a certain angle. The surface of the hanger cavity 1-6 has a rectangular hole 1-64 matched with the self-destructing fastener 1-83 on the rectangular pull hook 1-85. The front end of the cavity 1-61 are shrunk by two protection sheets 1-62 into a narrow slit 1-65, which can be passed through by the triangular pull hook 1-84 at the forefront of the plunger hanger 1-6. The narrow slit 1-65 is also the passageway of medicinal liquid. The front end of the hanger cavity 1-6 is a screw nut 1-63 matched with a thread 1-71 at the rear end of the needle holder 1-7.

As shown in FIG. 1-7, the screw nut 1-63 at the front end of the hanger cavity 1-6 has a hollow circular through hole 1-69, whose rear is shrunk by two protection sheets 1-62 into a narrow slit 1-65, which can be passed through by the triangular pull hook 1-84 at the forefront of the plunger hanger 1-6. The through hole 1-69 is a passageway of drug, and is also a cavity for rotation of the triangular pull hook 1-84.

As shown in FIG. 1-8, the thread 1-71 at the rear end of the needle holder 1-7 is matched with the screw nut 1-63 at the front end of the hanger cavity 1-6. The thread 1-71 is a hollow circular through hole.

As shown in FIGS. 1-6 and 1-8, there is a through hole 1-74 at the center of the needle holder 1-1. The middle of the needle holder 1-7 has a coned needle lock 1-73. Please also refer to FIGS. 1-1 and 1-2. The mounting hole 1-13 at the center of the bottom of the needle cylinder 1-1 is a circular hole, which can be passed through by the coned needle lock 1-73 of the needle holder 1-7 with a slight force. The needle holder piston 1-4 is of an elliptic cylindrical shape, whose outer diameter is matched with the inner diameter of the needle cylinder 1-1. The center of the needle holder piston 1-4 has a needle holder hole 1-41, which can tightly mount the needle holder 1-7. In order to prevent the needle holder piston 1-4 from slipping off when pulled back, the needle holder 1-7 has a stop sheet 1-72. The front end of the needle holder 1-7 is a thread 1-75 for installing a needle.

Please refer to FIGS. 1 and 2 again. The needle cap 1-16 and the needle cylinder 1-1 are incorporate, between which a weak destructing device 1-17 is provided. The end portion of the needle cap 1-16 has an opening matched with the circular hole 1-13 at the bottom of the needle cylinder 1-1. The needle cap 1-16 has a circular long hole for receiving the needle.

Back to FIG. 1, when the present invention is assembled, the needle holder piston 1-4 is first slipped into the screw nut 1-63 of the hanger cavity 1-6, the needle head is installed on the needle holder 1-7, and the needle holder 1-7 is then screwed for assembling them four together. Because the needle holder piston 1-4 is made of thermoplastic, elastic material, their connection is tight. Next, the assembled part is slipped into the front portion of the needle cylinder 1-1 from the rear of the needle cylinder 1-1 to let the coned needle lock 1-73 of the needle holder 1-7 protrude out of the bottom face of the needle cylinder 1-1 from the mounting hole 1-13 at the bottom of the needle cylinder 1-1 and be tightly fastened. The plunger piston 1-3 is slipped into the screw nut 1-24 at the front end of the plunger 1-2, and the plunger hanger l-8 is then screwed for similarly assembling them three together. Subsequently, the assembled part is then slipped into the assembled cylinder part. When in use, the needle cap 1-16 and the needle cylinder 1-1 are first separated, the plunger 1-2 is then pulled out to suck in medicinal liquid for injection. The use method is the same as that of existent syringes. After injection is finished, because of positioning function of an elliptic shape, the rectangular pull hook 1-85 of the plunger 1-2 will exactly align with the rectangular hole 1-66 of the hanger cavity 1-6 to let the pull hook 1-85 enter the cavity 1-61 of the hanger cavity 1-6 and the triangular pull hook 1-84 at the forefront end of the plunger hanger 1-8 enter the narrow slit 1-65 formed by the protection sheets 1-62 in the hanger cavity 1-6. The plunger 1-2 is then turned. Meanwhile, the weak ring 1-26 of the plunger 1-2 is destructed. The plunger hanger 1-8 also rotates a certain angle to let the self-destructing fastener 1-83 of the rectangular pull hook 1-85 enter the rectangular hole 1-64 at the bottom face of the hanger cavity 1-6 to accomplish self destruction. Meanwhile, there is a certain angle between the triangular pull hook 1-84 and the narrow slit 1-65. Subsequently, the plunger 1-2 is pulled outwards. Meanwhile, the plunger 1-2, the plunger hanger 1-8, the needle holder piston 1-4, the hanger cavity 1-6, the needle holder 1-7 and the needle are pulled into the needle cylinder 1-1 altogether. The plunger 1-2 is then broken with a force or the plunger 1-2 is reversed to screw open the connection screw between the plunger 1-2 and the plunger hanger 1-8, hence retreating the plunger 1-2. Other parts are remained in the needle cylinder to accomplish the object of destructive protection.

As shown in FIGS. 2-1, 2-2 and 2-3, a syringe according to a second embodiment of the present invention comprises a needle cylinder 2-1, a plunger 2-2, a plunger hanger 2-8, a hanger cavity 2-6, a needle holder lock 2-7 and a needle holder 2-67. A handle 2-21 is provided on one end of the plunger 2-2. The handle 2-21 has symmetric protection wings 2-29. The other end of the plunger 2-2 has a plunger piston 2-3. The handle 2-21 is engaged with the plunger piston 2-3 by a plunger body 2-22. There is a hole 2-13 on the center from bottom of the needle cylinder 2-1, which is used to mount the needle holder lock 2-7. There is a hole 2-12 at the edge from bottom of the needle cylinder 2-1, which is used to mount the needle holder 2-67. The needle holder lock 2-7 and the hanger cavity 2-6 whose rear is connected therewith by a thread are mounted on the needle cylinder 2-1 by a through hole 2-41 of the plunger piston 2-4. The hanger cavity 2-6 and the needle holder 2-67 are incorporate. The needle holder 2-67 is mounted on the needle cylinder 2-1 by the through hole 2-41 at the edge of the needle holder piston 2-4. The plunger piston holder 2-25 has a ring-shaped destructing device 2-26. The needle cap 2-16 and the needle cylinder 2-1 are incorporate, on which a weak destructing device 2-17 is provided. The weak destructing device 2-17 is destructed before use to accomplish the object of disposable use.

As shown in FIG. 2-3a, the cross-section of the plunger body 2-22 is a crossed structure. A triangular gap 2-23 is at the connection position of the four faces of the crossed structure of the plunger body 2-22 and the piston holder 2-25. The gap 2-23 is also connected with the inner circle of the ring-shaped destructing device 2-26. The gap 2-23 can also be of any shape like rectangular.

As shown in FIG. 2-3b, the destructing device on the piston holder 2-25 of the plunger body 2-22 is realized by providing a weak ring 2-26 on the elliptic piston holder 2-25.

As shown in FIGS. 2-3a and 2-3b, small columns 2-27 on the piston holder 2-25 of the plunger body 2-22 are used to limit the position relationship between the plunger piston 2-3 and the plunger 2-2.

As shown in FIGS. 2-2a, 2-2b and 2-2c, the cross section of the needle cylinder 2-1 is elliptic. The end portion of the needle cylinder 2-1 has an expanded hand support 2-11. The bottom center of the needle cylinder 2-1 has a hole 2-13 for mounting the needle holder lock 2-73. The bottom edge of the needle cylinder 2-1 has a hole 2-12 for mounting the needle holder 2-67. The front end of the inner wall of the needle cylinder 2-1 has ring-shaped protuberances for enhancing the firmness of a needle holder piston 2-4. The needle cap 2-16 and the needle cylinder 2-1 are incorporate, between which a weak destructing device 2-17 provided.

As shown in FIGS. 2-1, 2-3 and 2-4, the front end of the piston holder 2-25 of the plunger 2-2 has a screw nut 2-24 engaged with the plunger hanger 2-8. The rear of the plunger hanger 2-8 has a thread 2-81 matched with the screw nut 2-24. The middle of the plunger hanger 2-8 has a projective circular disk 2-82 for fastening the plunger piston 2-3. The front end of the plunger hanger 2-8 has a rectangular pull hook 2-85 matched with the hanger cavity 2-6. The rectangular pull hook 2-85 has a triangular pillared fastener 2-83 having self-destructing function. The forefront end of the plunger hanger 2-8 is a triangular pull hook 2-84.

As shown in FIG. 2-5, the plunger piston 2-3 has a through hole 2-32 to be slipped into by the screw nut 2-24 at the front end of the plunger 2-2. The plunger piston 2-3 is of an elliptic cylindrical shape. The plunger piston 2-3 has a circular hole 2-31 matched with the small column 2-27 on the piston holder 2-25. The plunger piston 2-3 can be made of thermoplastic, elastic material. The diameter of the through hole 2-32 is slightly smaller than that of the screw nut 2-24. External force can be exploited to slip the screw nut 2-24 into the through hole 2-32 to ensure firm installation of the plunger piston 2-3.

As show in FIG. 2-7, the surface of the hanger cavity 2-6 has a rectangular hole 2-66 similar to the rectangular pull hook 2-85. The rectangular hole 2-66 sticks into the hanger cavity 2-6 a certain depth to form a cavity 2-61, which can let the rectangular pull hook 2-85 rotate a certain angle. The surface of the hanger cavity 2-6 has a rectangular hole 2-64 matched with the self-destructing fastener 2-83 on the rectangular pull hook 2-85. The front end of the cavity 2-61 are shrunk by two protection sheets 2-62 into a narrow slit 2-65, which can be passed through by the triangular pull hook 2-84 at the forefront of the plunger hanger 2-6. The narrow slit 2-65 is also the passageway of medicinal liquid. The front end of the hanger cavity 2-6 is a screw nut 2-63 matched with a thread 2-71 at the rear end of the needle holder 2-7.

As also shown in FIG. 7, the screw nut 2-63 at the front end of the hanger cavity 2-6 has a hollow circular through hole 2-69, whose rear is shrunk by two protection sheets 2-62 into a narrow slit 2-65, which can be passed through by the triangular pull hook 2-84 at the forefront of the plunger hanger 2-6. The through hole 2-69 is a passageway of drug, and is also a cavity for rotation of the triangular pull hook 2-84.

As shown in FIG. 2-8, the thread 2-71 at the rear end of the needle holder 2-7 is a hollow through hole matched with the screw nut 2-63 at the front end of the hanger cavity 2-6. The front end of the through hole is closed by a stop sheet 2-72. The front end of the needle holder lock 2-7 is a coned needle holder fastener 2-73.

As shown in FIGS. 2-6, 2-7 and 2-8, there is a through hole 2-74 for medicinal liquid at the center of the needle holder 2-67. The middle of the needle holder 2-67 has a circular needle lock 2-60. The needle holder 2-67 is engaged with the hanger cavity 2-6 by a thread. Please also refer to FIGS. 2-1 and 2-2. The mounting hole 2-12 at the bottom edge of the needle cylinder 2-1 is a circular hole, which can be passed through by the circular needle lock 2-60 of the needle holder 2-67 with a slight force. The mounting hole 2-13 at the bottom center of the needle cylinder 2-1 is a circular hole, which can be passed through by the circular needle holder fastener 2-73 of the needle holder lock 2-7 with a slight force. The needle holder piston 2-4 is of an elliptic cylindrical shape, whose outer diameter is matched with the inner diameter of the needle cylinder 2-1. The center of the needle holder piston 2-4 has a needle holder fastening hole 2-41, which can tightly mount the needle holder lock 2-7. The edge of the needle holder piston 2-4 has a needle holder hole 2-42, which can tightly mount the needle holder 2-67. In order to prevent the needle holder piston 2-4 from slipping off when the needle holder lock 2-7 is pulled back, the needle holder 2-7 has a stop sheet 2-72 thereon.

As shown in FIGS. 2-1 and 2-2, the needle cap 2-16 and the needle cylinder 2-1 are incorporate, between which a weak destructing device 2-17 is provided. The end portion of the needle cap 2-16 has an opening matched with the mounting hole 2-12 at the bottom of the needle cylinder 2-1. The needle cap 2-16 has a long circular hole for receiving the needle therein.

Back to FIG. 2-1, when the present invention is assembled, the needle holder piston 2-4 is first slipped into the screw nut 2-63 of the hanger cavity 2-6 and the needle holder 2-67, and the needle holder lock 2-7 is then screwed for assembling them four together. Because the needle holder piston 2-4 is made of thermoplastic, elastic material, their connection is tight. Next, the assembled part is slipped into the front portion of the needle cylinder 2-1 from the rear of the needle cylinder 2-1 to let the circular needle fastener 2-73 of the needle holder lock 2-7 protrude out of the bottom face of the needle cylinder 2-1 from the mounting hole 2-13 at the bottom center of the needle cylinder 2-1 and be tightly fastened. Subsequently, the plunger piston 2-3 is slipped into the screw nut 2-24 at the front end of the plunger 2-2, and the plunger hanger 2-8 is then screwed for similarly assembling them three together. The assembled part is then slipped into the assembled cylinder part.

FIGS. 2-9 to 2-15 show a syringe according to another embodiment of the present invention.

As shown in FIG. 9, one end of a plunger 2-2′ has a handle 2-21′. Two ends of the handle 2-21′ have symmetric protection wings 2-29′. The other end of the plunger 2-2′ has a circular plate 2-28′ transversally placed at the end portion thereof. Two symmetric semi-elliptic piston holders 2-25′ are provided at two edges of a crossed structure and outside the circular plate 2-28′. The outer edge of the two piston holders 2-25′ is an elliptic shape in accordance with the elliptic inner wall of the needle cylinder 2-1′. The piston holders 2-25′ form point connection structures with the two edges of the crossed structure, hence forming destructing devices 2-26′ on the piston holders 2-25′. When the plunger is turned, the two connection points of the piston holders are destructed to accomplish self-destructing object. There is a screw rod 2-24′ engaged with the plunger hanger provided at the center of the front end of the circular plate 2-28′ of the plunger. Two small columns 2-27′ are symmetrically arranged on the two semi-elliptic piston holders 2-25′. The positions of the small columns 2-27′ correspond to those of circular holes 2-31′ on the rear end face of the plunger piston 2-3′. The small columns 2-27′ are used to limit the position relationship between the plunger piston 2-3′ and the plunger 2-2′.

Please refer to FIGS. 2-9a to 2-9e. The cross-section of the plunger body 2-22′ is a crossed structure. The breakable region on the plunger body 2-22′ is triangular gaps on the four faces of the crossed structure at the middle of the plunger body 2-22′. Other breakable regions can also be used according to the real situation. For instance, the breakable region on the plunger body 2-22′ can be breakable gaps of other shapes at predetermined positions of the plunger body 2-22′.

As shown in FIG. 2-10, the distal end of the plunger hanger 2-8′ has an inner thread 2-81′ connected with the plunger 2-2′. The middle of the plunger hanger 2-8′ has a projective circular disk 82′ for fastening the plunger piston 2-3′. The front end of the plunger hanger 2-8′ has an arrowheaded triangular pull hook 2-84′ matched with the hanger cavity 2-6′. A prismatic pillared self-destructing fastener 2-83′ parallel to the central line is provided at the outside of the front end face of the projective circular disk 2-82′ near the pull hook 2-84′.

As shown in FIGS. 2-13 and 2-10, the hanger cavity 2-6′ is elliptic, and has a rectangular hole 2-65′ with the same shape as the axial projection of the arrowheaded triangular pull hook 2-84′ at the center of one side near the pull hook. The rectangular hole 2-65′ can be passed through by the triangular pull hook 2-84′. The front end of the hanger cavity 2-6′ is a screw nut 2-63′ matched with a thread 2-71′ at the rear end of the needle holder 2-7′. An inner screw hole 2-69′ of the screw nut 2-63′ is a cavity for rotation of the triangular pull hook 2-84′. An integrally formed needle holder 2-67′ is provided on the hanger cavity 2-6′. The center of the needle holder 2-67′ has a through hole 2-68′ for medicinal liquid. The one side of the hanger cavity 2-6′ near the pull hook has an annular pillar-shaped groove 2-64′ of about 90 degrees. The shape of the axial projection of the annular pillar-shaped groove 2-64′ is clockwise arrowheaded. The width of the annular pillar-shaped groove 2-64′ gradually diminishes from the start end to the distal end thereof, and ends with a triangular arrowhead at the distal end. As shown in FIG. 2-10, the prismatic pillared self-destructing fastener 2-83′ of the plunger hanger 2-8′ matches with the arrowheaded annular pillar-shaped groove 2-64′ of the hanger cavity 2-6′. The plunger hanger 2-8′ is turned to let the prismatic self-destructing fastener 2-83′ slide from the start end of the arrowheaded annular pillar-shaped groove 2-64′ into the arrowhead of the diminishing arrowheaded groove to be stuck in the arrowhead and not able to recede, hence accomplishing self-destructing object.

As shown in FIG. 2-11, the plunger piston 2-3′ has a through hole 2-32′, which can be slipped into by the outer circle at the rear end of the plunger hanger 2-8′. The plunger piston 2-3′ is elliptic. The plunger piston 2-3′ has a hole 2-31′ matched with the small column 2-27′ on the semicircular plunger piston holder 2-25′. Both the circular hole 2-31′ and the plunger piston 2-3′ can be made of thermoplastic, elastic material. The diameter of the through hole 2-32′ is slightly smaller than that of the outer circle at the rear end of the plunger hanger 2-8′. External force can be exploited to slip the outer circle at the rear end of the plunger hanger 2-8′ into the through hole 2-32′ to ensure firm installation of the plunger piston 2-3′.

As shown in FIG. 2-12, the needle holder piston 2-4′ is of an elliptic cylindrical shape, whose outer diameter is matched with the inner diameter of the needle cylinder 2-1′. The center of the needle holder piston 2-4′ has a needle holder fastening hole 2-41′, which can tightly mount the needle holder lock 2-7′. The edge of the needle holder piston 2-4′ has a needle holder hole 2-42′, which can tightly mount the needle holder 2-67′.

The needle cylinder 2-1′ can adopt the structure shown in FIG. 2-2.

As shown in FIG. 2-14, an outer thread 2-71′ at one end of the needle holder lock 2-7′ is connected with the screw nut 2-63′ of the hanger cavity 2-6′. In order to prevent the needle holder piston 2-4′ from sliding off when the needle holder lock. 2-7′ is pulled back, the needle holder lock 2-7′ has a stop sheet 2-72′ thereon. The needle holder lock 2-7′ has also a coned needle holder fastener 2-73′ at the front end thereof.

When this embodiment of the present invention is assembled, the needle holder 2-7′ first passes through the needle holder piston 2-4′ to be tightly assembled with the hanger cavity 2-6′ using the thread, is installed at the bottom of the needle cylinder 2-1′ from the rear opening of the needle cylinder 2-1′. The needle holder lock 2-7′ matches with the mounting hole 2-13′ at the center of the needle cylinder 2-1′. The medicinal liquid passageway matches with the mounting hole 2-12 for medicinal liquid of the needle cylinder 2-1′. Next, after the plunger 2-2′ is assembled with the plunger piston 2-3′, the small column of the plunger matches with the small cylindrical hole of the plunger piston, is tightly engaged with the plunger hanger 2-8′ by the thread, and is installed from the rear opening of the needle cylinder until tightly contacting with part of the hanger cavity. When in use, after injection is finished, the prismatic pillar 2-83′ of the plunger hanger tightly contacts the arrowheaded groove 2-64′ of the hanger cavity, the triangular pillared pull hook 2-84′ on the pull hook 2-8′ exactly passes through the rectangular hole 2-65′ in the hanger cavity. The plunger is then turned 90 degrees clockwise (the breakable point on the plunger is destructed at this time). Meanwhile, the triangular pillared pull hook 2-84′ on the plunger hanger exactly hangs the two long sides of the rectangular hole 2-65′ of the hanger cavity 2-6′. Simultaneously, the prismatic pillar 2-83′ on the plunger hanger passes through the arrowheaded groove 2-64′ gradually diminishing with the rotation locus and forcibly enters the end portion of the arrowhead, hence letting the prismatic pillar be not able to recede. Accordingly, the triangular pillared pull hook 2-84′ on the plunger hanger cannot rotate, hence accomplishing self-destructing function.

As shown in FIG. 3-1, a syringe of the present invention comprises a needle cylinder 3-1, a plunger 3-2, a needle holder 3-7 and a needle 3-5. A handle 3-21 is provided on one end of the plunger 3-2. Two ends of the handle 3-21 have symmetric protection wings 3-29. The other end of the plunger 3-2 has a plunger piston 3-3. The handle 3-21 is engaged with the plunger piston 3-3 by a plunger body 3-22. The needle holder 3-7 is mounted on the needle cylinder 3-1 by a needle holder piston 3-4 located at the bottom of the needle cylinder 3-1. A needle cap 3-6 and the needle cylinder 3-1 are incorporate, on which a weak destructing device 3-61 is provided. The destructing device 3-61 is destructed before use to accomplish the object of disposable use. The plunger body 3-22 has a breakable region. The breakable region is openings at the middle of the plunger body 3-22 or breakable grooves at predetermined portions of the plunger body 3-22. The breakable region breaks after the syringe is used to accomplish the object of destructive protection.

As shown in FIGS. 3-3a, 3-3c and 3-3d, the cross-section of the plunger body 3-22 is a crossed structure. Openings 3-23 of the breakable region on the plunger body 3-22 are on the four faces of the crossed structure at the middle the plunger body 3-22. Other breakable regions can also be used according to the real situation. For instance, the breakable region on the plunger body 3-22 can be a breakable gap at a predetermined position of the plunger body 1-22.

The openings 3-23 are rectangular, whose area is 30% to 80% of the area of the cross section of the plunger body 3-22.

Please refer to FIGS. 3-2a, 3-2b and 3-2d. The needle cylinder 3-1 is of an elliptic cylindrical shape, whose end portion has an expanded hand support.

As shown in FIG. 3-3, the bottom end of the plunger body 3-22 is engaged with a circular piston holder 3-25, whose center has a piston shaft 3-28 for slipping into the plunger piston 3-3. The piston shaft 3-28 has a circular disk 3-26 for fixing the plunger piston 3-3. Please refer to FIG. 3-3. The plunger piston 3-3 has a through hole 3-33, which can be slipped into by the piston shaft 3-28 and the circular disk 3-26. The plunger piston 3-3 is similar to an elliptic cylindrical shape. The center of the elliptic cylindrical face of the plunger piston 3-3 is recessed, and one bottom face of the plunger piston 3-3 protrudes toward the center. The front end of the plunger piston 3-3 has a rectangular opening 3-31. The plunger piston 3-3 can be made of thermoplastic, elastic material. The through hole 3-33 is slightly smaller than the piston shaft 3-28 and the circular disk 3-26. External force can be exploited to slip the piston shaft 3-28 and the circular disk 3-26 into the through hole 3-33 to ensure firm installation of the plunger piston 3-3. The bottom end of the piston shaft 3-28 has a groove 3-24 so that a rear spacer 3-8 can be assembled easily. The rear spacer 3-8 has a slot 3-82 and an opening 3-81 thereon.

As shown in FIG. 3-7, the needle holder 3-7 has a ring-shaped needle lock 3-72 for installing the needle head of the syringe. The center of the needle holder 3-7 has a through hole 3-74. The middle of the needle holder 3-7 has a pair of sector stop sheets 3-75. The circular arc angle of the sector stop sheet 3-75 is preferred to be less than 45 degrees. The circular arc angle chosen in the figure is 30 degrees. The bottom of the needle holder 3-7 has a lock disk 3-71. Please also refer to FIGS. 3-1 and 3-2. The central circular hole 3-13 can be passed through by the needle lock 3-72 of the needle holder 3-7. The circular are angle of the sector hole 3-14 is at least not smaller than that of the stop sheets 3-75 of the needle holder 3-7 so that the pair of stop sheets 3-75 can successfully pass through. The circular arc angle shown in the figure is 45 degrees, which is larger than that of the stop sheets 3-75. As also shown in FIG. 3-4, the bottom of the needle cylinder has a needle holder piston 3-4 for installing the needle holder. The needle holder piston 3-4 is of an elliptic cylindrical shape, whose outer diameter matches the inner diameter of the needle cylinder. The center of the needle holder piston 3-4 has a needle holder hole, which can tightly mount the expanded portion of the lock disk 3-71.

As shown in FIG. 3-5, the needle holder hole comprises an end portion hole 3-43 passed through by the lock disk 3-71 and the lock tooth 3-27, a middle hole 3-42 for receiving and locking the lock disk 3-71 and a bottom hole 3-41 in accordance with the middle of the needle holder.

As shown in FIGS. 3-1 and 3-3, the piston shaft 3-28 protrudes out of the plunger piston 3-3. The end portion of the part of the piston shaft protruding out of the plunger piston has a pair of lock teeth 3-27. The lock teeth 3-27 are fan-shaped, and are symmetric with respect to its central axis. The circular arc angle of the sector lock teeth is generally smaller than 45 degrees. The circular arc angle of the sector lock teeth 3-27 shown in the figure is 30 degrees. Please also refer to FIGS. 3-7a, 3-7c and 3-7d. The bottom face of the lock disk 3-71 has a pair of sector openings 3-76 similar to and slightly larger than the lock teeth 3-27 at the end portion of the plunger piston. The circular arc angle of the sector openings 3-76 shown in the figure is 45 degrees. The lock teeth 3-27 can be inserted into the openings 3-76. The openings 3-75 stick into the lock disk 3-71 a certain depth and rotates and expands around its center to form a cavity 3-73 for receiving the lock teeth 3-27. The cavity 3-73 can let the lock teeth 3-27 rotate an angle not smaller than the circular arc angle of the lock teeth. The circular arc angle of the cavity 3-73 shown in the figure is 90 degrees.

As shown in FIGS. 3-1, 3-2 and 3-5, a pair of outer walls in the same direction and adjacent to the sector hole 3-14 have recessed portions 3-12. The recessed portions 3-12 are of the shape of a quarter ellipse with a circular arc angle larger than that of the stop sheets so that the stop sheets can be turned to fix the needle holder after the needle holder is installed at the bottom of the needle cylinder.

Please further refer to FIGS. 3-1 and 3-2. The needle cap 3-6 and the needle cylinder 3-1 are incorporate, between which a weak destructing device 3-61 is provided. The end portion of the destructing device 3-61 has an opening matched with the circular hole 3-13 at the bottom of the needle cylinder 3-1. The opening has a long circular hole for receiving the needle 3-5 therein.

Back to FIG. 1, when the present invention is assembled, the needle holder 3-7 is first slipped into the needle holder piston 3-4. Meanwhile, the lock disk 3-71 is exactly placed in the central hole 3-42 of the needle holder hole. Because the needle holder piston is made of thermoplastic, elastic material, their connection is tight. Next, the assembled needle holder 3-7 is slipped into the bottom of the needle cylinder from the top opening of the needle cylinder 3-1 to let the sector stop sheets 3-75 protrude out of the bottom face of the needle cylinder 3-1 from the mounting hole at the bottom of the needle cylinder and rotate so that the sector stop sheets 3-75 can be placed in the recessed portions 3-12 at the bottom of the needle cylinder. The assembled plunger 3-2 is then slipped thereinto. When in use, the needle cap 3-6 and the needle cylinder 3-1 are first separated, the plunger 3-2 is then pulled out to suck in medicinal liquid for injection. The use method is the same as that of existent syringes. After injection is finished, because of positioning function of an elliptic shape, the lock teeth 3-27 will exactly align with the sector openings 3-76 on the lock disk to let the lock teeth 3-27 enter the needle holder cavity 3-73. The needle holder 3-7 is then turned a certain angle to let the sector stop sheets 3-75 of the needle holder align with the sector holes 3-14 at the bottom of the needle cylinder. Meanwhile, the lock teeth 3-27 will enter the lock groove because of rotation of the needle holder cavity 3-73. Subsequently, the plunger 3-2 is pulled outwards. Meanwhile, the plunger 3-2, the needle holder piston 3-4, the needle holder 3-7 and the needle 3-5 enter the needle cylinder 3-1 altogether. The plunger 3-2 is then broken with a force to accomplish the object of destructive protection.

Although the present invention has been described with reference to the preferred embodiments thereof, it will be understood that the invention is not limited to the details thereof. Various substitutions and modifications have been suggested in the foregoing description, and others will occur to those of ordinary skill in the art. Therefore, all such substitutions and modifications are intended to be embraced within the scope of the invention as defined in the appended claims.

I claim:

1. A self-destructing syringe comprising a needle cylinder with an elliptic cross section, a plunger, a plunger piston, a needle holder and a needle, said plunger piston being provided at the front end of said plunger, a handle of said plunger-being engaged with said plunger piston by a plunger body, said self-destructing syringe further comprising a plunger hanger, a hanger cavity and a needle holder piston, there being a mounting hole at the bottom center of said needle cylinder for mounting said needle holder, said needle holder and said hanger cavity whose rear is engaged therewith being mounted on said needle cylinder by said needle holder piston at the bottom in said needle cylinder, the front end of said plunger being engaged with said plunger hanger by a thread, a hook portion at the front end of said plunger hanger sticking into a cavity of said hanger cavity, a self-destructing structure being provided between said plunger hanger and said hanger cavity, said plunger body having a breakable region thereon, a needle cap and said needle cylinder being incorporate, on which a weak destructing device is provided.

2. The self-destructing syringe as claimed in claim 1, wherein the cross section of said plunger body is a crossed structure, the breakable region on said plunger body is triangular breakable grooves on four faces of said crossed structure at the middle of said plunger body, and the area of said breakable region is 30% to 80% of the area of the cross section of said plunger body.

3. The self-destructing syringe as claimed in claim 1, wherein a plunger piston holder with an elliptic cross section is provided at the front end of said plunger body, said plunger piston holder has a ring-shaped destructing device and small columns for fixing said elliptic plunger piston, said plunger piston has circular holes corresponding to said small columns, a screw nut engaged with sad plunger hanger is provided at the front end of said plunger piston holder, said plunger piston has a through hole matched with said screw nut, a thread matched with said screw nut is provided at the rear of said plunger hanger, a projective circular disk for fastening said plunger piston is provided at the middle of said plunger hanger, a rectangular pull hook matched with said hanger cavity is provided at the front end of said plunger hanger, said rectangular pull hook has a self-destructing fastener, a triangular pull hook is provided at the forefront end of said plunger hanger.

4. The self-destructing syringe as claimed in claim 1, 2 or 3, wherein part of said plunger hanger protrudes out of said plunger piston, the end portion of said plunger hanger protruding out of said plunger piston has a rectangular pull hook, which is symmetrically arranged with respect to its central axis, a rectangular opening similar to said rectangular pull hook is provided at the bottom face of said hanger cavity, said rectangular opening sticks into said hanger cavity a certain depth to form a cavity, which can let said rectangular pull hook rotate a certain angle, a rectangular hole matched with said self-destructing fastener on said rectangular pull hook is provided at the bottom face of said hanger cavity, the front end of said cavity is shrunk by two protection sheets into a narrow slit, which can be passed through by a triangular pull hook at the forefront end of said plunger hanger, said narrow slit is also a passageway of medicinal liquid, a screw nut matched with a thread at the rear end of said needle holder is provided at the front end of said hanger cavity, said needle holder piston has a through hole matched with said screw nut.

5. The self-destructing syringe as claimed in claim 4, wherein there is a through hole at the center of said needle holder, a coned needle lock is provided at the middle of said needle holder, said mounting hole at the bottom center of said needle cylinder is a circular hole, which can be passed through by said coned needle lock of said needle holder with a slight force, said needle holder piston is of an elliptic cylindrical shape, whose outer diameter is matched with the inner diameter of said needle cylinder, there is a needle holder hole at the center of said needle holder piston, which can tightly mount said needle holder, said needle holder has a stop sheet to prevent said needle holder piston from slipping off when being pulled back, a needle is fixedly connected at the front end of said needle holder.

6. The self-destructing syringe as claimed in claim 5, wherein ring-shaped protuberances for enhancing firmness of said needle holder piston are provided on the inner wall of said needle cylinder, an opening matched with said mounting hole at the bottom of said needle cylinder is provided at the end portion of said needle cap, said needle cap has a long circular hole for receiving said needle therein.

7. The self-destructing syringe as claimed in claim 2, wherein a plate transversally placed at the end portion of said plunger body is provided at the front end thereof, two symmetric semi-elliptic piston holders are provided at two opposite edges of said crossed structure and outside said plate, said piston holders achieve point connection with the two edges of said crossed structure to form a destructing device on said piston holders, two small pillars are provided on said two semi-elliptic piston holders, circular holes corresponding to said small pillars are provided on the rear end face of said plunger piston.

8. The self-destructing syringe as claimed in claim 7, wherein the outer circle of the rear end of said plunger hanger is matched with a through hole of said plunger piston, a projective circular disk for fastening said plunger piston is provided at the middle of said plunger hanger, a triangular pull hook matched with said hanger cavity is provided at the front end of said plunger hanger, a prismatic pillared self-destructing fastener parallel to the central line is provided at the outer side of the front end face of said projective circular disk near said pull hook, said hanger cavity is of an elliptic shape matched with an inner elliptic hole of said needle holder piston, a rectangular opening having the same shape as the axial projection of said triangular pull hook of said plunger hanger and capable of being passed through by said triangular pull hook is provided at the center of the one side of said hanger cavity near said pull hook, said rectangular opening is also a passageway of medicinal liquid, a screw nut matched with a thread at the rear end of said needle holder is provided at the front end of said hanger cavity, an inner screw hole of said screw nut is a passageway of medicinal liquid and is also a cavity for rotation of said triangular pull hook, an annular pillared groove of about 90 degrees is provided at the one side of said hanger cavity near said pull hook, the shape of the axial projection of said annular pillared groove is arrowheaded, the width of said annular pillared groove gradually diminishes from the start end to the distal end thereof and ends with a prismatic pillared arrowhead at the distal end, which can exactly receive said prismatic pillared self-destructing fastener, said prismatic pillared self-destructing fastener of said plunger hanger matches with said arrowheaded annular pillared groove of said hanger cavity.

9. The self-destructing syringe as claimed in claim 8, wherein said self-destructing fastener of said plunger hanger can be two coned pull hooks at the front end of said plunger hanger, two coned holes matched with said coned pull hooks of said plunger hanger are provided at the rear end face of said hanger cavity, a pillared hole smaller than the maximum outer diameter of said coned pull hook is provided at the outer end of said coned hole, and a cone having the same shape as said coned pull hook is provided at the inner end thereof.

10. A self-destructing syringe capable of replacing its needle head comprising a needle cylinder with an elliptic cross section, a plunger, a plunger hanger, a hanger cavity and a needle holder, a handle being provided on one end of said plunger, symmetric protection wings being provided at one end of said handle, a plunger piston being provided at the other end of said plunger, said handle being engaged with said plunger piston by a plunger body, there being a mounting hole for mounting said needle holder at the bottom center of said needle cylinder, said needle holder and said hanger cavity whose rear is connected therewith by a thread being mounted on the needle cylinder by said plunger piston located on the bottom of said needle cylinder, said plunger body having a breakable region, the cross section of said plunger body being a crossed structure, said breakable region being breakable grooves at-predetermined portions of the middle of said plunger body, weak points being provided on said plunger body to let said destructing device of said plunger piston holder take effect, a needle cap and said needle cylinder being incorporate, between which a weak destructing device is provided, there being a through hole at the center of said needle holder, a circular tube-shaped needle lock being provided at the middle of said needle holder, said mounting hole at the bottom center of the needle cylinder being a circular hole, which can be passed through by said circular tube-shaped needle lock of said needle holder with a slight force, said needle holder piston being of an elliptic cylindrical shape, whose outer diameter is matched with the inner diameter of said needle cylinder, there being a needle holder hole at the center of said needle holder piston, which can tightly mount said needle holder, said needle holder having a stop sheet to prevent said needle holder piston from slipping off when being pulled back, a thread for installing a needle being provided at the front end of said needle holder.

11. The self-destructing syringe as claimed in claim 10, wherein the cross section of said plunger body is a crossed structure, said breakable region on said plunger body is on four faces of said crossed structure of the middle of said plunger body, said breakable region being triangular, whose area is 30%˜80% of the area of the cross section of said plunger body, a triangular gap of said plunger body being provided at the connection position of the four faces of said crossed structure of said plunger body and said piston holder, said gap being engaged with the inner circle of said ring-shaped destructing device.

12. The self-destructing syringe as claimed in claim 10, wherein the front end of said plunger body is engaged with said piston holder having an elliptic cross section, said piston holder has a ring-shaped destructing device and small columns for fixing said elliptic piston, said plunger piston has circular holes corresponding to said small columns, a screw nut engaged with said plunger hanger is provided at the front end of said plunger body, a thread matched with said screw nut is provided at the rear of said plunger hanger, a projective circular disk for fastening said plunger piston is provided at the middle of said plunger hanger, a rectangular pull hook matched with said hanger cavity is provided at the front end of said plunger hanger, said rectangular pull hook has a self-destructing fastener, a triangular pull hook is provided at the forefront end of said plunger hanger.

13. The self-destructing syringe as claimed in claim 10, 11 or 12, wherein part of said plunger hanger protrudes our of said plunger piston, a rectangular pull hook is provided at the end portion of said plunger hanger protruding out of said plunger piston, said rectangular pull hook is symmetrically arranged with respect to its central axis, a rectangular opening similar to said rectangular pull hook is provided at the bottom face of said hanger cavity, said rectangular opening sticks into said hanger cavity a certain depth to form a cavity, which can let said rectangular pull hook rotate a certain angle, a circular hole matched with said self-destructing fastener on said rectangular pull hook is provided at the bottom face of said hanger cavity, the front end of said cavity is shrunk by two protection sheets into a narrow slit, which can be passed through by a triangular pull hook at the forefront end of said plunger hanger, said narrow slit is also a passageway of medicinal liquid, a screw nut matched with a thread at the rear end of said needle holder is provided at the front end of said hanger cavity, said needle holder piston has a through hole matched with said screw nut.

14. The self-destructing syringe as claimed in claim 10, wherein the cross section of said needle cylinder is elliptic, there is a mounting hole at the bottom of said needle cylinder, ring-shaped protuberances for enhancing firmness of said needle holder piston are provided on the inner wall of said needle cylinder, said needle cap and said needle cylinder are incorporate, between which a weak destructing device is provided, an opening matched with the mounting hole at the bottom of said needle cylinder is provided at the end portion of said needle cap, a long circular hole for receiving said needle is provided at the inside of said needle cap.

15. A self-destructing intravenous injection syringe comprising a needle cylinder with an elliptic cross section, a plunger, a plunger piston, a needle holder and a needle holder lock, a plunger piston being provided at the front end of said plunger, a handle of said plunger being engaged with said plunger piston by a plunger body, said self-destructing syringe further comprising a plunger hanger and a hanger cavity, there being a hole at the bottom center of said needle cylinder for mounting said needle holder lock, there also being a hole at the bottom edge of said needle cylinder for mounting said needle holder, said needle holder lock and said hanger cavity whose rear is engaged therewith being mounted on said needle cylinder by said needle holder piston, said needle holder being provided on said hanger cavity and integrally formed with said hanger cavity, said needle holder being mounted on said needle cylinder by a mounting hole at the edge of said needle holder piston, the cross section of said plunger body being a crossed structure, said plunger having a breakable region, a needle cap and said needle cylinder being incorporate, on which a weak destructing device is provided.

16. The self-destructing syringe as claimed in claim 15, wherein a piston holder with an elliptic cross section is connected at the font end of said plunger body, said piston holder has a ring-shaped destructing device and small columns for fixing said elliptic piston, said breakable region on said plunger body is gaps provided at the connection position of said plunger body and said piston holder, said gaps are engaged with the inner circle of said ring-shaped destructing device, said plunger piston has circular holes corresponding to said small columns, a screw nut engaged with sad plunger hanger is provided at the front end of said plunger piston holder, said plunger piston has a through hole matched with said screw nut, a thread matched with said screw nut is provided at the rear of said plunger hanger, a projective circular disk for fastening said plunger piston is provided at the middle of said plunger hanger, a rectangular pull hook matched with said hanger cavity is provided at the front end of said plunger hanger, said rectangular pull hook has a self-destructing fastener, a triangular pull hook is provided at the forefront end of said plunger hanger.

17. The self-destructing syringe as claimed in claim 16, wherein part of said plunger hanger protrudes out of said plunger piston, a rectangular pull hook is provided at the front end of said plunger hanger protruding out of said plunger piston, said rectangular pull hook is symmetrically arranged with respect to its central axis, a rectangular opening similar to said rectangular pull hook is provided at the surface of said hanger cavity, said rectangular opening sticks into said hanger cavity a certain depth to form a cavity, which can let said rectangular pull hook rotate a certain angle, a rectangular hole matched with said self-destructing fastener on said rectangular pull hook is provided at the surface of said hanger cavity, the front end of said cavity is shrunk by two protection sheets into a narrow slit, which can be passed through by a triangular pull hook at the forefront end of said plunger hanger, a screw nut matched with a thread at the rear end of said needle holder lock is provided the front end of said hanger cavity, said needle holder piston has a through hole matched with said screw nut and another through hole matched with said needle holder.

18. The self-destructing syringe as claimed in claim 17, wherein a hollow thread is provided at the rear end of said needle holder lock, the front end of said needle holder lock is closed by a stop sheet, a coned needle holder fastener is provided at the front end of said needle holder lock, said mounting hole at the bottom center of said needle cylinder is a circular hole, which can be passed through by said coned needle fastener of said needle holder lock with a slight force, said mounting hole at the bottom edge of said needle cylinder is used to mount said needle holder, said needle holder piston is of an elliptic cylindrical shape, whose outer diameter is matched with the inner diameter of said needle cylinder, there is a through hole at the center of said needle holder piston, which can tightly mount said needle holder lock, there is another through hole at the edge of said needle holder piston, which matches said needle holder and can tightly mount said needle holder, said needle holder has a stop sheet to prevent said needle holder piston from slipping off when being pulled back.

19. The self destructing syringe as claimed in claim 18, wherein said needle cylinder is elliptic, said mounting hole at the bottom center of said needle cylinder matches said needle holder fastener, said mounting hole at the bottom edge of said needle cylinder matches said needle holder, ring-shaped protuberances for enhancing firmness of said needle holder piston are provided on the inner wall of said needle cylinder, said needle cap and said needle cylinder are incorporate, between which a weak destructing device is provided, an opening matched with said mounting hole at the bottom center of said needle cylinder is provided at the end portion of said needle cap, a long circular hole for receiving said needle is provided at the inside of said needle cap.

20. The self-destructing syringe as claimed in claim 15, wherein a plate transversally placed at the end portion of said plunger body is provided at the front end thereof, two symmetric semi-elliptic piston holders are provided at two opposite edges of said crossed structure and outside said plate, the outer profile of said two piston holders is in accordance with the inner elliptic wall of said needle cylinder, said piston holders achieve point connection with the two edges of said crossed structure to form a destructing device on said piston holders, two small pillars are provided on said two semi-elliptic piston holders, holes corresponding to said small pillars are provided on the rear end face of said plunger piston.

21. The self-destructing syringe as claimed in claim 20, wherein the front end of said plunger is engaged with the distal end of said plunger hanger by a thread, the outer circle of the rear end of said plunger hanger is matched with a through hole of said plunger piston, a projective circular disk for fastening said plunger piston is provided at the middle of said plunger hanger, a triangular pull hook matched with said hanger cavity is provided at the front end of said plunger hanger, a prismatic pillared self-destructing fastener parallel to the central line is provided at the outer side of the front end face of said projective circular disk near said pull hook, said hanger cavity is of an elliptic shape matched with the inner elliptic hole of said needle holder piston, a rectangular opening having the same shape as the axial projection of said triangular pull hook of said plunger hanger and capable of being passed through by said triangular pull hook is provided at the center of the one side of said hanger cavity near said pull hook, said rectangular opening is also a passageway of medicinal liquid, a screw nut matched with a thread at the rear end of said needle holder is provided at the front end of said hanger cavity, an inner screw hole of said screw nut is a passageway of medicinal liquid and is also a cavity for rotation of said triangular pull hook, an annular pillared groove of about 90 degrees is provided at the one side of said hanger cavity near said pull hook, the axial projection of said annular pillared groove is arrowheaded, the width of said annular pillared groove gradually diminishes from the start end to the distal end thereof and ends with a prismatic pillared arrowhead at the distal end, which can exactly receive said prismatic pillared self-destructing fastener, said prismatic pillared self-destructing fastener of said plunger hanger matches said arrowheaded annular pillared groove of said hanger cavity.

22. A self-destructing syringe comprising a needle cylinder with an elliptic cross section, a plunger, a needle holder and a needle, a handle being provided at one end of said plunger, symmetric protection wings being provided at two ends of said handle, a plunger piston being provided at the other end of said plunger, said handle being engaged with said plunger piston by a plunger body, there being a mounting hole at the bottom center of said needle cylinder for mounting said needle holder, said needle holder being mounted on said needle cylinder by said needle holder piston at the bottom in said needle cylinder, said plunger body having a breakable region, the cross section of said plunger body being a crossed structure, said breakable region being openings at the middle of said plunger body or breakable grooves at predetermined portions of said plunger body, said needle being tightly matched with said needle holder by a connection structure at the front end of said needle holder, a needle cap and said needle cylinder being incorporate, on which a weak destructing device is provided.

23. The self-destructing syringe as claimed in claim 22, wherein the cross section of said plunger body is a crossed structure, said breakable region on said plunger body is rectangular openings on four faces of said crossed structure at the middle of said plunger body, and the area of said openings is 30% to 80% of the area of the cross section of said plunger body.

24. The self-destructing syringe as claimed in claim 22, wherein the bottom end of said plunger body is engaged with a circular piston holder, a piston shaft for slipping into said elliptic piston is provided at the center of said piston holder, said piston shaft has a projective circular disk for fixing said piston, a wing disk matched with said needle holder is provided at the front end of said piston shaft.

25. The self-destructing syringe as claimed in claim 22, 23 or 24, wherein said needle holder has a ring-shaped needle lock for installing a needle head of said syringe, said ring-shaped needle lock has a thread therein, there is a through hole at the center of said needle holder, a pair of sector stop sheets are provided at the middle of said needle holder, a lock disk is provided at the bottom of said needle holder, said mounting hole at the bottom center of said needle cylinder comprises a central circular hole passed through by said needle lock of said needle holder and a pair of sector holes symmetric to the central axis and connected with said circular hole and passed through by said sector stop sheets, said needle holder piston is of an elliptic cylindrical shape, whose outer diameter matches the inner diameter of said needle cylinder, there is a needle holder hole at the center of said needle holder piston, an expanded portion capable of tightly mounting said lock disk is provided at the middle of said needle holder hole, a front spacer is provided at the front end of said needle holder piston when assembled.

26. The self-destructing syringe as claimed in claim 25, wherein the circular arc angle of said sector stop sheets is smaller than 30 degrees, and the circular arc angle of said sector holes is smaller than 45 degrees.

27. The self-destructing syringe as claimed in claim 25, wherein said piston shaft of said plunger protrudes out of said piston, a pair of lock teeth are provided at the end portion of said piston shaft protruding out of said piston, said lock teeth are fan-shaped and symmetric to its central axis, a pair of sector openings similar to and slightly larger than said lock teeth at the end portion of said plunger piston are provided at the bottom face of said lock disk, said openings stick into said lock disk a certain depth and rotates and expands around its center to form a cavity for receiving said lock teeth, a groove matched with a rear spacer is provided at the bottom of said piston shaft.

28. The self-destructing syringe as claimed in claim 27, wherein the circular arc angle of said fan-shaped lock teeth is smaller than 30 degrees, the circular arc angle of said fan-shaped openings is smaller than 45 degrees, and the circular arc angle of said cavity is smaller than 90 degrees.

29. The self-destructing syringe as claimed in claim 25, wherein said needle cylinder is elliptic, a pair of sector holes are provided at the bottom of said needle cylinder, recessed portions are provided at outer walls of said needle cylinder in the same direction and adjacent to said sector holes, said recessed portions are of the shape of a quarter ellipse.

30. The self-destructing syringe as claimed in claim 22, wherein said needle cap and said needle cylinder are incorporate, between which a weak destructing device is provided, an opening matched with said circular hole at the bottom of said needle cylinder is provided at the end portion of said needle cap, a long circular hole for receiving said needle is provided at the inside of said needle cap.
